SEQUENCE LISTING (2 sequences)

SEQ ID NO: 1 - DNA, 12 bp, Artificial Sequence
  Description of Artificial Sequence Synthetic oligonucleotide
gaaagtgaaagt

SEQ ID NO: 2 - protein, 6 aa, Artificial Sequence
  Description of Artificial Sequence Synthetic 6xHis tag
HHHHHH

(19) United States
(12) Patent Application Publication
(10) Pub. No.: US 2022/0195501 A1
(43) Pub. Date: Jun. 23, 2022

(54) Targeted Sequence Detection by Nanopore Sensing of Synthetic Probes

(71) Applicant: Nooma Bio, Inc., Santa Cruz, CA (US)

(72) Inventors: Trevor J. Morin, Santa Cruz, CA (US); Daniel Alexander Heller, Santa Cruz, CA (US)

(21) Appl. No.: 17/693,222

(22) Filed: Mar. 11, 2022

Related U.S. Application Data
(63) Continuation of application No. 15/513,472, filed on Mar. 22, 2017, now abandoned.
(60) Provisional application No. 62/056,378, filed on Sep. 26, 2014.

(51) Int. Cl. C12Q 1/6825; C12Q 1/6816

(52) U.S. Cl. CPC C12Q 1/6825; C12Q 2600/154; C12Q 2537/164; C12Q 1/6816

(57) ABSTRACT

Disclosed herein are methods and compositions for detection of one or more specific sequences of polynucleotides in a solution using a nanopore. In some embodiments, methods and compositions for identifying a polynucleotide in a sample or for target sequence detection of a polynucleotide are disclosed herein.

### CROSS REFERENCE TO RELATED APPLICATION

This application is a continuation of U.S. application Ser. No. 15/513,472 filed Mar. 22, 2017, which is a 35 U.S.C. 371 national stage filing of International Application No. PCT/US2015/052729, filed on Sep. 28, 2015, which claims the benefit of U.S. Provisional Patent Application No. 62/056,378 filed Sep. 26, 2014, all of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Mar. 11, 2022, is named 52005US_CRF_sequencelisting.txt and is 798 bytes in size.

### FIELD OF THE INVENTION

The invention relates to methods and compositions for target sequence detection using a nanopore device.

### BACKGROUND

Detection, localization, and copy number determinations of specific sequence regions within a stretch of nucleic acids, referred to here as “target sequence detection,” has applications in biomedical science and technology, medicine, agriculture and forensics, as well as in other fields. The detection of genes and their modifications, sequence, location, or number, is important for the advancement of molecular diagnostics in medicine. DNA microarrays, PCR, Southern Blots, and FISH (Fluorescent in situ Hybridization) are all methods that can be used to perform or aid in target sequence detection. These methods are slow and labor intensive, and have limited accuracy and resolution. More recent methods, such as real-time PCR and next-generation sequencing (NGS) technologies, have improved throughput, but still do not have sufficient resolution for many applications.

Solid-state nanopores have been demonstrated to detect molecules by applying a voltage across the pores, and measuring current impedance as the molecules pass through the nanopore. The overall efficacy of any given nanopore device depends on its ability to accurately and reliably measure current impedance and to distinguish among different types of molecules that pass through. Experiments published in literature have demonstrated both the detection of DNA and RNA strands passing through the pores, and synthetic molecules that hybridize to specific sequences on them. However, no one has been able to use these to generate a high throughput and reliable nanopore device for detecting probes on specific DNA or RNA sequences. Probes developed to date have been insufficient for reliable sequence detection. Therefore, what is needed is a set of probes and probe complexes capable of sequence-specific binding for detection in a nanopore.

### SUMMARY

Provided herein are methods of detecting a polynucleotide comprising a target sequence in a sample, comprising: contacting said sample with a probe that specifically binds to said polynucleotide comprising said target sequence under conditions that promote binding of said probe to said target sequence to form a polynucleotide-probe complex; loading said sample into a first chamber of a nanopore device, wherein said nanopore device comprises at least one nanopore and at least said first chamber and a second chamber, wherein said first and second chamber are in electrical and fluidic communication through said at least one nanopore, and wherein the nanopore device further comprises an independently-controlled voltage across each of said at least one nanopores and a sensor associated with each of said at least one nanopores, wherein said sensor is configured to identify objects passing through the at least one nanopore, and wherein said polynucleotide-probe complex translocating through said at least one nanopore provides a detectable signal associated with said polynucleotide-probe complex; and determining the presence or absence of said polynucleotide-probe complex in said sample by observing said detectable signal, thereby detecting said polynucleotide comprising said target sequence. In an embodiment, the method further comprises generating a voltage potential through said at least one nanopore, wherein said voltage potential generates a force on said polynucleotide-probe complex to pull said polynucleotide-probe complex through said at least one nanopore, causing said polynucleotide-probe complex to translocate through said at least one nanopore to generate said detectable signal.

In some embodiments, said polynucleotide is DNA or RNA. In an embodiment, said detectable signal is an electrical signal. In an embodiment, said detectable signal is an optical signal. In an embodiment, said probe comprises a molecule selected from the group consisting of: a protein, a peptide, a nucleic acid, a TALEN, a CRISPR, a peptide nucleic acid, or a chemical compound. In an embodiment, said probe comprises a molecule selected from the group consisting of: a deoxyribonucleic acid (DNA), a ribonucleic acid (RNA), a peptide nucleic acid (PNA), a DNA/RNA hybrid, polypeptide, or any chemically derived polymer.

In an embodiment, said probe comprises a PNA molecule bound to a secondary molecule configured to facilitate detection of the probe bound to said polynucleotide during translocation through said at least one nanopore. In a further embodiment, said secondary molecule is a PEG. In a further embodiment, said PEG has a molecular weight of at least 1 kDa, 2 kDa, 3 kDa, 4 kDa, 5 kDa, 6 kDa, 7 kDa, 8 kDa, 9 kDa, or 10 kDa.

In an embodiment, said method of detecting a polynucleotide comprising a target sequence in a sample further comprises applying a condition to said sample suspected to alter the binding interaction between the probe and the target sequence. In a further embodiment, the condition is selected from the group consisting of: removing the probe from the sample, adding an agent that competes with the probe for binding to the target sequence, and changing an initial pH, salt, or temperature condition.

In an embodiment, said polynucleotide comprises a chemical modification configured to modify binding of the polynucleotide to the probe. In a further embodiment, the chemical modification is selected from the group consisting of biotinylation, acetylation, methylation, summolation, glycosylation, phosphorylation and oxidation.

In an embodiment, said probe comprises a chemical modification coupled to the probe through a cleavable bond. In an embodiment, said probe interacts with the target sequence of the polynucleotide via a covalent bond, a hydrogen bond, an ionic bond, a metallic bond, van der Waals force, hydrophobic interaction, or planar stacking interactions. In an embodiment, said method of detecting a polynucleotide comprising a target sequence in a sample further comprises contacting the sample with one or more detectable labels capable of binding to the probe or to the polynucleotide-probe complex. In an embodiment, said polynucleotide comprises at least two target sequences.

In an embodiment, said nanopore is about 1 nm to about 100 nm in diameter, 1 nm to about 100 nm in length, and wherein each of the chambers comprises an electrode. In an embodiment, said nanopore device comprises at least two nanopores configured to control the movement of said polynucleotide in both nanopores simultaneously. In an embodiment, said method of detecting a polynucleotide comprising a target sequence in a sample further comprises reversing said independently-controlled voltage after initial detection of the polynucleotide-probe complex by said detectable signal, so that the movement of said polynucleotide through the nanopore is reversed after the probe-bound portion passes through the nanopore, thereby identifying again the presence or absence of a polynucleotide-probe complex.

In an embodiment, said nanopore device comprises two nanopores, and wherein said polynucleotide is simultaneously located within both of said two nanopores. In a further embodiment, said method of detecting a polynucleotide comprising a target sequence in a sample comprises comprising adjusting the magnitude and or the direction of the voltage in each of said two nanopores so that an opposing force is generated by the nanopores to control the rate of translocation of the polynucleotide through the nanopores.

Also provided herein is a method of detecting a polynucleotide or a polynucleotide sequence in a sample, comprising: contacting said sample with a first probe and a second probe, wherein said first probe specifically binds to a first target sequence of said polynucleotide under conditions that promote binding of said first probe to said first target sequence, wherein said second probe specifically binds to a second target sequence of said polynucleotide under conditions that promote binding of said second probe to said second target sequence; contacting said sample with a third molecule is configured to bind to said first and second probe simultaneously when said first and second probe are within a sufficient proximity to each other under conditions that promote binding of said third molecule to said first probe and said second probe, thereby forming a fusion complex comprising said polynucleotide, said first probe, said second probe, and said third molecule; loading said sample into a first chamber of a nanopore device, wherein said nanopore device comprises at least one nanopore and at least said first chamber and a second chamber, wherein said first and second chamber are in electrical and fluidic communication through said at least one nanopore, and wherein the nanopore device further comprises a controlled voltage potential across each of said at least one nanopores and a sensor associated with each of said at least one nanopores, wherein said sensor is configured to identify objects passing through the at least one nanopore, and wherein said fusion complex translocating through said at least one nanopore provides a detectable signal associated with said fusion complex; and determining the presence or absence of said fusion complex in said sample by observing said detectable signal.

In an embodiment, said polynucleotide is DNA or RNA. In an embodiment, said detectable signal is an electric signal. In an embodiment, said detectable signal is an optical signal. In an embodiment, said sufficient proximity is less than 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 300, 400, or 500 nucleotides. In an embodiment, said third molecule comprises a PEG or an antibody.

In an embodiment, said third molecule and said first and second probes are bound to ssDNA, and wherein said ssDNA linked to said third molecule comprises a region complementary to a region of ssDNA linked to said first probe and is complementary to a region of ssDNA linked to said second probe. In an embodiment, the method of detecting a polynucleotide or a polynucleotide sequence in a sample further comprising contacting the sample with one or more detectable labels capable of binding to the third molecule or to the fusion complex.

Also provided herein is a kit comprising a first probe, a second probe, and a third molecule, wherein the first probe is configured to bind to a first target sequence on a target polynucleotide, wherein the second probe is configured to bind to a second target sequence on said target polynucleotide, and wherein said third molecule is configured to bind to the first probe and the second probe when said first and second probes are bound to said polynucleotide at said first and second target sequences, thereby locating the first and second probe in sufficient proximity to allow binding of said third molecule to said first and second probes simultaneously.

In an embodiment, said first probe and said second probe are selected from the group consisting of: a protein, a peptide, a nucleic acid, a TALEN, a CRISPR, a peptide nucleic acid, or a chemical compound. In an embodiment, said third molecule comprises a PEG or an antibody. In an embodiment, said third molecule comprises a modification to modify binding affinity to said probes.

Also provided herein is a nanopore device comprising at least two chambers and a nanopore, wherein said device comprises a modified PNA probe bound to a polynucleotide within said nanopore.

Also provided herein is a dual-pore, dual-amplifier device for detecting a charged polymer through two pores, the device comprising an upper chamber, a middle chamber and a lower chamber, a first pore connecting the upper chamber and the middle chamber, and a second pore connecting the middle chamber and the lower chamber, wherein said device comprises a modified PNA probe bound to a polynucleotide within said first or second pore.

In an embodiment, the device is configured to control the movement of said charged polymer through both said first pore and said second pore simultaneously. In an embodiment, the modified PNA probe is bound to at least one PEG molecule. In an embodiment, the device further comprises a power supply configured to provide a first voltage between the upper chamber and the middle chamber, and provide a second voltage between the middle chamber and the lower chamber, each voltage being independently adjustable, wherein the middle chamber is connected to a common ground relative to the two voltages, wherein the device provides dual-amplifier electronics configured for independent voltage control and current measurement at each pore, wherein the two voltages may be different in magnitude, wherein the first and second pores are configured so that the charged polymer is capable of simultaneously moving across both pores in either direction and in a controlled manner.

### BRIEF DESCRIPTION OF THE DRAWINGS

Provided as embodiments of this disclosure are drawings which illustrate by exemplification only, and not limitation.

FIG. 1 illustrates the detection of a target molecule bound to a modified probe in pair of nanopores as one embodiment of the presently disclosed method.

FIG. 2 shows the effect of probe binding to a target molecule on the electrical signal generated when the complex translocates through a nanopore.

FIG. 3A and FIG. 3B each show an embodiment with two probes bound to a polynucleotide at their respective target sequences, and a third bridging molecule (e.g, an antibody) to facilitate probe detection in a nanopore when both probes are bound to the scaffold.

FIG. 4 shows two probes bound to a polynucleotide at their respective target sequences, wherein the third bridging molecule is PEG and attaches to the probes via complementary ssDNA linkers to enable probe detection when both probes are bound to the scaffold in sufficient proximity.

FIG. 5 shows two probes bound to the polynucleotide at their respective target sequences in sufficient proximity to allow detection of an optical signal generated due to their proximity, e.g., through Forster resonance energy transfer (FRET).

FIG. 6A is a schematic of a system that combines a nanopore device with an epifluorescence microscope to enable detection of a fluorophore modified binding agent.

FIG. 6B is an illustration of what is seen through the detector as the fluorophore passes through an in-plane two nanopore device. FIG. 6C shows the change in the current amplitude and the corresponding fluorescent signal when a scaffold passes through the nanopore.

FIG. 7 shows binding of probes that have groups (e.g., fluorophores) that are cleavable, to aid in detection.

FIG. 8 illustrates the multiplex capability of the present technology by including probes of differing size that each bind to a unique target sequence in the target-bearing molecule. In this illustration, double-stranded DNA is the polynucleotide with a target sequence and multiple different DNA binding probes that bind to target sequences that are desired to be detected.

FIG. 9A shows a PNA ligand that has been modified as to increase ligand charge, and therefore facilitate detection by a nanopore. FIG. 9B shows an example in which a double-stranded DNA is used as the target bearing polymer and multiple different DNA binding probes that bind to target sequences that are desired to be detected.

FIG. 10 shows multiple distinct sequence-specific probes bound to DNA as it transverses through a nanopore to allow for multiplexed detection.

FIGS. 11A-C shows a nanopore and representative current signatures and populations from translocations of molecules through the nanopore. In FIG. 11A, a solid state pore and voltage path is shown. FIG. 11B shows the current blockade and dwell time of a molecule passing through a nanopore. FIG. 11C shows distinguishing populations of molecules passing through a nanopore based on their dwell time and mean current amplitude.

FIG. 12A shows an example of the use of PNA probes bound to biotin that complex with a larger neutravidin molecule to allow detection of sequences on the DNA scaffold.

FIG. 12B shows the binding sites for the PNA probes on the DNA scaffold.

FIG. 13 shows translocation of unbound DNA, free neutravidin, and complexed PNA-Biotin bound to DNA and to neutravidin. The resulting current signatures (current on y-axis, time on x-axis) when the molecule translocates through a nanopore under an applied voltage for each complex are also shown.

that the DNA/PNA/Neutravidin complexes cause translocation current signatures that are detectable above other background event types (e.g., unbound DNA alone, and Neutravidin alone) and can therefore be tagged as detectable PNA probes bound to DNA (i.e. DNA/PNA/Neutravidin complex) events.

FIG. 14A shows a scatter plot of events characterized by duration and mean conductance shift due to translocation through the nanopore in three populations, DNA alone (x), Neutravidin alone (square), and DNA complexed with a biotin probe attached to neutravidin (circle). FIG. 14B shows a histogram of dwell time probability associated with each of the three populations described above. FIG. 14C shows a gel shift assay of DNA only (lane 2), a sample comprising DNA, PNA with 3 biotin sites to bind neutravidin, and neutravidin (lane 3), a sample comprising DNA, PNA with 7 biotin sites to bind neutravidin, and neutravidin (lane 3), a sample comprising DNA, PNA with 16 biotin sites to bind neutravidin, and neutravidin (lane 3), and a sample comprising DNA, PNA with 36 biotin sites to bind neutravidin, and neutravidin (lane 3).

FIG. 15 shows a diagram of probe binding sites on a DNA scaffold, where the probe is VspR protein.

FIG. 16A shows a diagram of an unbound DNA molecule passing through a nanopore, and the representative current signature associated with a single molecule passing through a nanopore. FIG. 16B shows a diagram of a VspR-bound DNA molecule passing through a nanopore, and the representative current signature associated with it's passing through the nanopore.

FIG. 17 shows ten more representative current attenuation events consistent with the VspR-bound scaffold passing through the pore

FIG. 18A shows a PNA-PEG probe bound to its target sequence on a dsDNA molecule. FIG. 18B shows the results of a gel shift assay with the following samples: DNA only (lane 1), DNA/PNA (lane 2), DNA/PNA-PEG (10 kDa) (lane 3), and DNA/PNA-PEG (20 kDa) (lane 4). FIG. 18C shows the results of a gel shift assay with the following samples: DNA marker (lane 1), random DNA sequence incubated with PNA probe (lane 2), DNA with single mismatch at target sequence incubated with corresponding PNA probe (lane 3), and DNA with target sequence mixed with corresponding PNA probe specific to the target sequence (lane 4).

FIG. 19A shows representative current signature events as the molecule depicted below each current signature translocates through the nanopore under an applied voltage.

FIG. 19B shows a scatter plot of events characterized by duration and mean conductance shift due to translocation through the nanopore in three populations: DNA/bisPNA (square), DNA/bisPNA-PEG 5 kDa (circle), and DNA/bisPNA-PEG 10 kDa (diamond). FIG. 19C shows a histogram of mean conductance shift probability associated with each of the three populations described above. FIG. 19D shows a histogram of event duration probability associated with each of the three populations described above.

FIG. 20A shows representative event signatures correlated with the translocation of a PNA-PEG probe bound to a DNA molecule. FIG. 20B shows the mean conductance shift v. duration plot for each recorded event in the nanopore from a sample comprising bacterial DNA and PNA-PEG probe. FIG. 20C and FIG. 20D show corresponding histograms to characterize these events detected by mean conductance shift and duration of each event respectively. FIG. 20E shows the results of a gel shift assay showing: 100 bp ladder (lane 1), 300 bp DNA with wild type cftr sequence incubated with the PNA-PEG probe (lane 2), and 300 bp DNA with the cftr ΔF508 sequence incubated with the PNA-PEG probe (lane 3).

FIG. 21A shows the results of the gel shift assay, with lane 1 comprising S. mitis bacterial DNA without a bisPNA-PEG bound, and lane 2 comprising S. mitis DNA with a site-specific bisPNA-PEG bound. FIG. 21B shows a scatter plot of mean conductance shift (dG) on the vertical axis vs. duration on the horizontal axis for all recorded events in the two consecutive experiments. The first sample included bacterial DNA with PEG-modified PNA probes (DNA/bisPNA-PEG). The second sample included bacterial DNA alone.

Some or all of the figures are schematic representations for exemplification; hence, they do not necessarily depict the actual relative sizes or locations of the elements shown. The figures are presented for the purpose of illustrating one or more embodiments with the explicit understanding that they do not limit the scope or the meaning of the claims that follow below.

### DETAILED DESCRIPTION

Throughout this application, the text refers to various embodiments of the present nutrients, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

Also throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

As used in the specification and claims, the singular form “a,” “an” and “the” include plural references unless the context clearly dictates otherwise. For example, the term “an electrode” includes a plurality of electrodes, including mixtures thereof.

As used herein, the term “comprising” is intended to mean that the devices and methods include the recited components or steps, but not excluding others. “Consisting essentially of” when used to define devices and methods, shall mean excluding other components or steps of any essential significance to the combination. “Consisting of” shall mean excluding other components or steps. Embodiments defined by each of these transition terms are within the scope of this invention.

All numerical designations, e.g., distance, size, temperature, time, voltage and concentration, including ranges, are approximations which are intended to encompass ordinary experimental variation in measurement of the parameters, and that variations are intended to be within the scope of the described embodiment. It is to be understood, although not always explicitly stated that all numerical designations are preceded by the term “about”. It also is to be understood, although not always explicitly stated, that the components described herein are merely exemplary and that equivalents of such are known in the art.

The term “nanopore” (or, just “pore”) as used herein refers to a single nano-scale opening in a membrane that separates two volumes. The pore can be a protein channel inserted in a lipid bilayer membrane, for example, or can be engineered by drilling or etching or using a voltage-pulse method through a thin solid-state substrate, such as silicon nitride or silicon dioxide or graphene or layers of combinations of these or other materials. Geometrically, the pore has dimensions no smaller than 0.1 nm in diameter and no bigger than 1 micron in diameter; the length of the pore is governed by the membrane thickness, which can be sub-nanometer thickness, or up to 1 micron or more in thickness. For membranes thicker than a few hundred nanometers, the nanopore may be referred to as a “nano channel.”

As used here, the term “nanopore instruments” refers to devices that combine one or more nanopores (in parallel or in series) with circuitry for sensing single molecule events. Specifically, nanopore instruments use a sensitive voltage-clamp amplifier to apply a specified voltage across the pore or pores while measuring the ionic current through the pore(s). When a single charged molecule such as a double-stranded DNA (dsDNA) is captured and driven through the pore by electrophoresis, the measured current shifts, indicating a capture event (i.e., the translocation of a molecule through the nanopore, or the capture of a molecule in the nanopore), and the shift amount (in current amplitude) and duration of the event are used to characterize the molecule captured in the nanopore. After recording many events during an experiment, distributions of the events are analyzed to characterize the corresponding molecule according to its shift amount (i.e., its current signature). In this way, nanopores provide a simple, label-free, purely electrical single-molecule method for biomolecular sensing.

As used herein, the term “event” refers to a translocation of a detectable molecule or molecular complex through the nanopore and its associated measurement. It can be defined by its current, duration, and/or other characteristics of detection of the molecule in the nanopore. A plurality of events with similar characteristics is indicative of a population of molecules or complexes that are identical or have similar characteristics (e.g., bulk, charge).

Molecular Detection

The present disclosure provides methods and systems for molecular detection and quantitation. In addition, the methods and systems can also be configured to measure the affinity of a probe binding to a target molecule. Further, such detection, quantitation, and measurement can be carried out in a multiplexed manner, greatly increasing its efficiency.

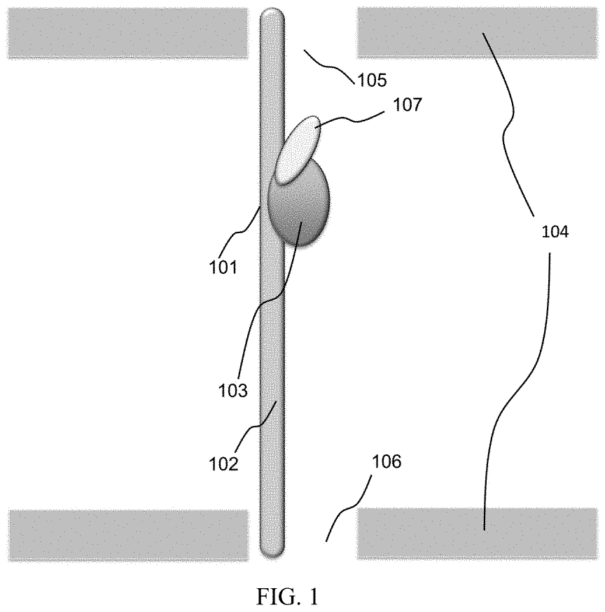

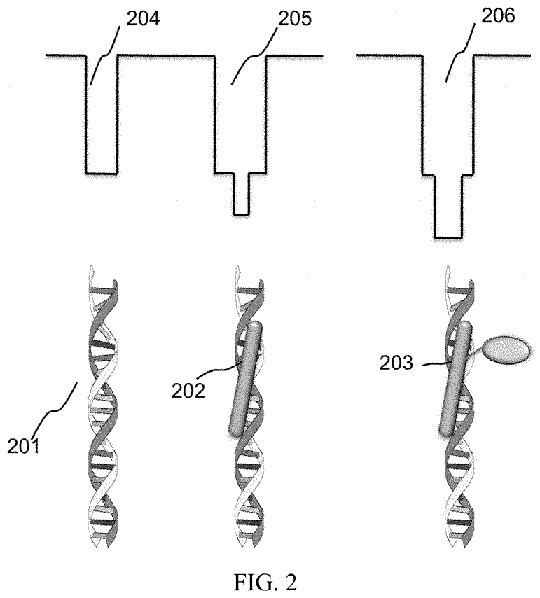

FIG. 1 provides an illustration of one embodiment of the disclosed methods and systems. More specifically, the system includes a target bearing molecule (102) that contains a target motif 101 that is desired to be detected or quantitated. The probe (103) is capable of binding to a specific binding motif 101 on the target bearing molecule 102. An additional molecule can be added to aid detection of the probe (107) if present on the target bearing polynucleotide.

Therefore, if all present in a solution, the probe 103 binds to the target motif through the specific recognition of the probe for the target motif 101. Such binding causes the formation of a complex that includes the probe and the target sequence.

The formed complex (101/103 or 101/103/107) can be detected by a device (104) that includes two pores (105 and 106) that separates an interior space of the device into 3 volumes, and a sensor adjacent to the pore configured to identify objects passing through the pore. This embodiment is a dual nanopore device with two nanopores in series. In some embodiments, the nanopore device includes electronic components to deliver controlled voltages across the nanopores (which voltages can, in some embodiments, be independently controlled and clamped) along with circuitry for measuring current flow across the nanopores. The voltages can be tuned to move in a controlled manner the polynucleotide from one volume to another across the pores. The polynucleic acid is charged or modified to contain charges, the applied potential or voltage differential across the pores facilitates and controls the movement of the charged scaffold through application of an electrostatic force on the charged molecule exposed to the voltage field. While FIG. 1 shows a dual nanopore device, the principles described above can be applied in other embodiments of the present invention using a single nanopore device. Unless specified below, references to a pore or nanopore or nanopore device are intended to encompass single, dual or multi-pore devices within the spirit of the present invention.

When a sample that includes the formed complex is loaded to the nanopore, the nanopore can be configured to pass the target bearing molecule through the pore. When the target motif is within the pore or adjacent to the pore, the binding status of the target motif can be detected by the sensor.

The “binding status” of a target motif, as used herein, refers to whether the binding motif is occupied by probe. Essentially, the binding status is either bound or unbound. Either, (i) the target motif is free and not bound to a probe (see 201 and 204 in FIG. 2), (ii) the target motif is bound to a probe, (see 202 and 205 in FIG. 2). Additionally, probes of different sizes or having different probe binding sites can be used to give additional current profiles (see, e.g., 203 and 206 in FIG. 2) to enable more than one target sequence to be detected on one target bearing molecule.

Detection of the binding status of a target motif can be carried out by various methods. In one aspect, by virtue of the different sizes of the target motif at each status (i.e. occupied or unoccupied), when the target motif passes through the pore, the different sizes result in different currents across the pore. In this respect, no separate sensor is required for the detection, as the electrodes, which are connected to a power source and can detect the current, can serve the sensing function. The two electrodes, therefore, can serve as a “sensor.”

In some aspects, an agent (e.g., 107 in FIG. 1) is added to the complex to add detection. This agent is capable of binding to the probe or polynucleotide/probe complex. In one aspect, the agent includes a charge, either negative or positive, to facilitate detection. In another aspect, the agent adds size to facilitate detection. In another aspect, the agent includes a detectable label, such as a fluorophore.

In this context, an identification of a bound status (ii) indicates that a target sequence in a target bearing molecule and is complexed with the probe. In other words, the target sequence is detected.

In another embodiment, bound molecules are spaced apart to individually detect bound molecules by impedance changes, wherein each bound molecule gives an impedance value that is not masked by neighboring bound molecules.

In one embodiment, bound probes are separated by a distance of at least 1 nm (i.e., approximately 3 bp for a nucleic acid-based polynucleotide). In another embodiment, the bound probes are separated by a distance of at least 10 nm (i.e., approximately 33 bp for a nucleic acid-based polynucleotide). In another embodiment, the bound probes are separated by a distance of at least 100 nm (i.e., approximately 333 bp for a nucleic acid-based polynucleotide). In another embodiment, the bound probes are separated by a distance of at least 500 nm (i.e., approximately 1666 bp for a nucleic acid-based polynucleotide).

In some aspects, the method further comprises having two independent probes that, if close enough to each other once bound to the polynucleotide, can bind a third molecule. Binding of this third molecule provides a different translocation current signature, thus providing evidence that the two independent probes are in close proximity.

Mechanisms to determine if probes are binding in close proximity allows us to use short probes that can distinguish between single base pair mismatches, and therefore detect alleles with single nucleotide polymorphism (or single nucleotide mutations), and longer probes to act as markers to establish a unique spot in the genome. In one embodiment, we use the two probes in combination to determine if a particular sequence is associated with a particular target gene. In another embodiment, this method is used to determine structural rearrangements by using probes as markers for regions in the genome. In another embodiment, we use the two probes in combination to determine if a particular sequence is chemically (epigenetically, e.g. methylation, hydroxymethylation) modified and associated with a particular target gene.

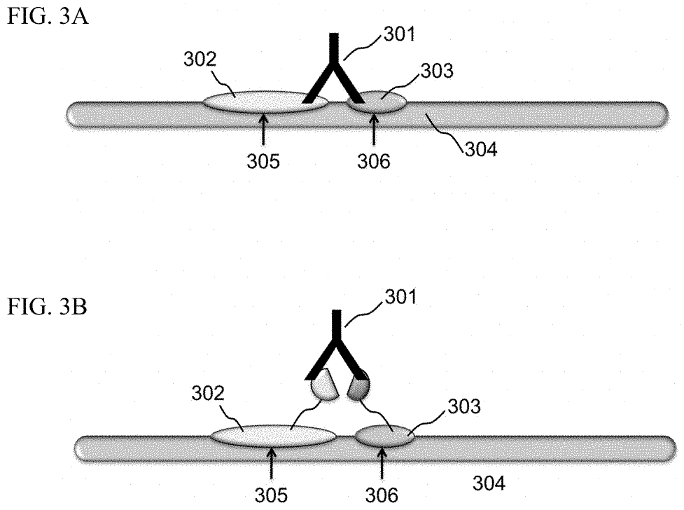

In one embodiment, the third molecule is an antibody (301) that only binds to the probes (305 and 306) if at least two probes are bound to the polynucleotide (304) and significantly close to each other (0.01 nm-50 nm) (FIG. 3A). In another embodiment, half of the epitope for the antibody (301) is connected to each probe molecule (302 and 303) (via covalent attachment, ionic, H-bond, or otherwise) causing antibody binding to be dependent on both epitopes being situated in close proximity, which indicates that the probes are near enough to each other (FIG. 3B). In one embodiment, each probe is a PNA molecule, and each PNA molecule comprises or is attached to a segment of a binding epitope for an antibody (covalent attachment, ionic, H-bond, or otherwise). In this embodiment, the partial epitopes must be in sufficient proximity for the antibody to bind to form a complex with the polynucleotide.

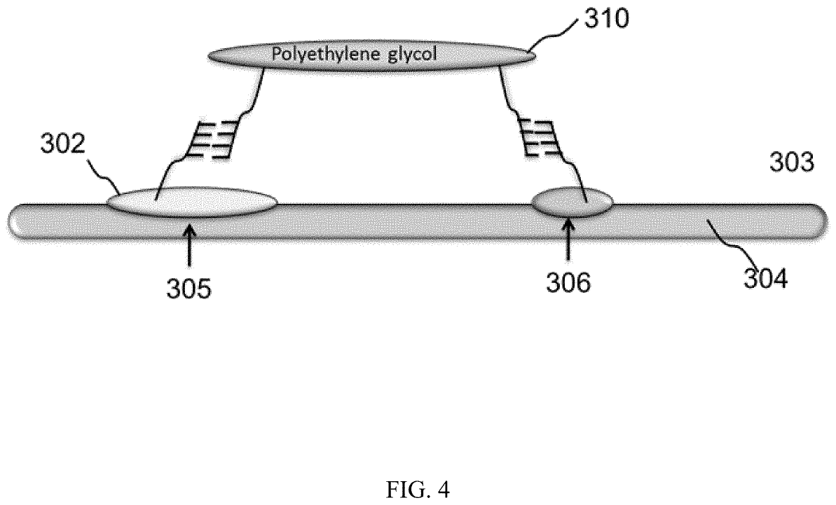

In another embodiment, a PEG molecule (310) is used as a third molecule to bind to two probes (302 and 303) in close proximity and bound to the polynucleotide (304). In some embodiments, the PEG is modified to provide sufficient bulk, charge, or other features that allow a unique signature when present (FIG. 4). In some embodiments, the PEG is modified to increase binding affinity for the probes that are in close proximity. This binding modification on a PEG can be, e.g., a single-stranded DNA (ssDNA) molecule at each end of the PEG that is complementary to free ssDNA attached to each probe. In an embodiment, the energy barrier required for PEG binding to the probes is only satisfied when both ssDNA oligos are bound to their complementary sequence attached to the probes (FIG. 4). Thus, probes that are at a distance that the PEG spans are detected in the nanopore through the detection of a PEG/probe/polynucleotide complex, while those farther apart are not. As one of ordinary skill will recognize ssDNA can be substituted by synthetic nucleic acid analogs such as PNAs or by RNAs.

In some aspects, two independent probes are modified to allow detection if they are bound to the polynucleotide in close proximity. In one embodiment, the modification to the probes comprises altering the ionic charge of the probes to alter the current signature when the probes are bound to the polynucleotide in close proximity and pass through the nanopore, as distinguished from the current signature when a single probe/polynucleotide complex passes through the nanopore without being in close proximity to a second probe. In one embodiment, adding positive charge to both probes (e.g. by labeling probes with 2-hydroxyethylthiosulfonate (MTSET)) provides a different translocation current signature when both probes are sufficiently close in space along the polynucleotide and the effect of charge is additive, as opposed to them binding to the polynucleotide far apart.

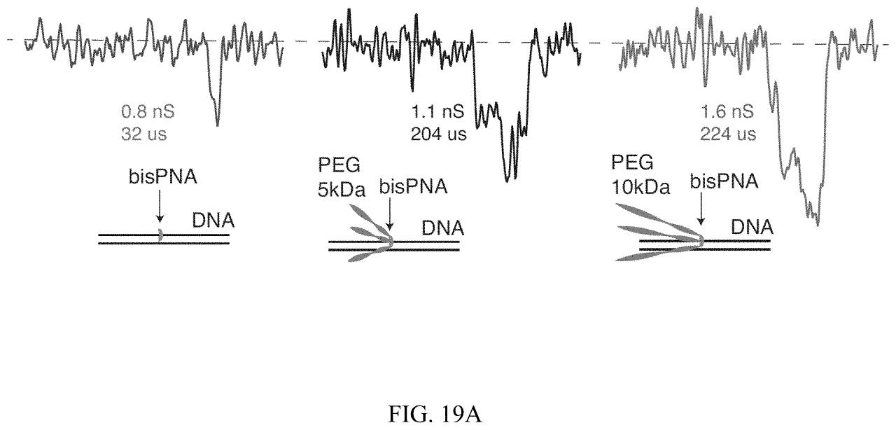

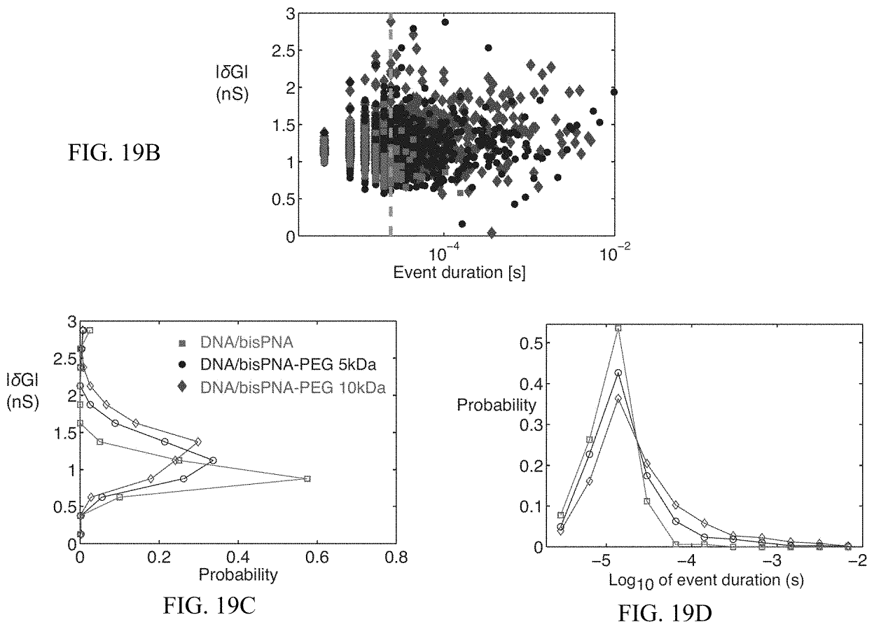

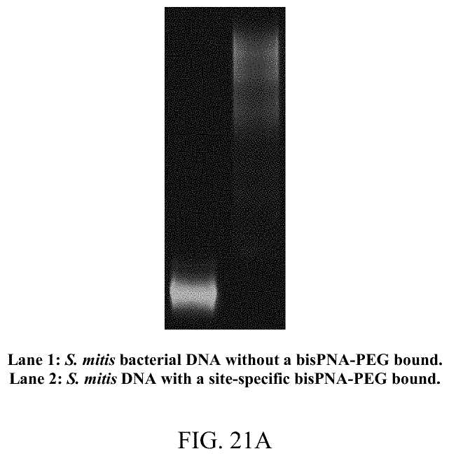

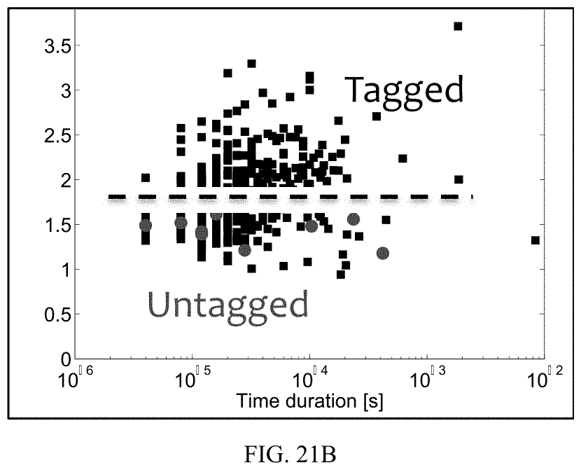

In some aspects, the method further comprises using probes that are sufficiently long as to enable binding to only one unique sequence in the target population, but also have the ability to not bind to the target site if only a single base pair mismatch is present. This is possible when using PNA probes. As shown (Strand-Invasion of Extended, Mixed-Sequence B-DNA by γPNAs, G. He, D. Ly et. al., J Am Chem Soc. 2009 Sep. 2; 131(34): 12088-12090. doi:10.1021/ja900228j) a 20 bp gamma-PNA probe is able to efficiently bind to a perfectly matched target sequence, but binding is abrogated when the target sequence and probe sequence differ by only one base. When considering the human genome that contains 3.1 billion bases, a 20 base pair sequence is likely to randomly occur 0.003 times. Thus, a 20 base pair probe designed to bind to a specific sequence under investigation is very unlikely to bind to an undesired location and provide a false positive. Examples contained within (FIGS. 19c and 21) show PNA and PNA-PEG probes selectively bind only complementary sequence.

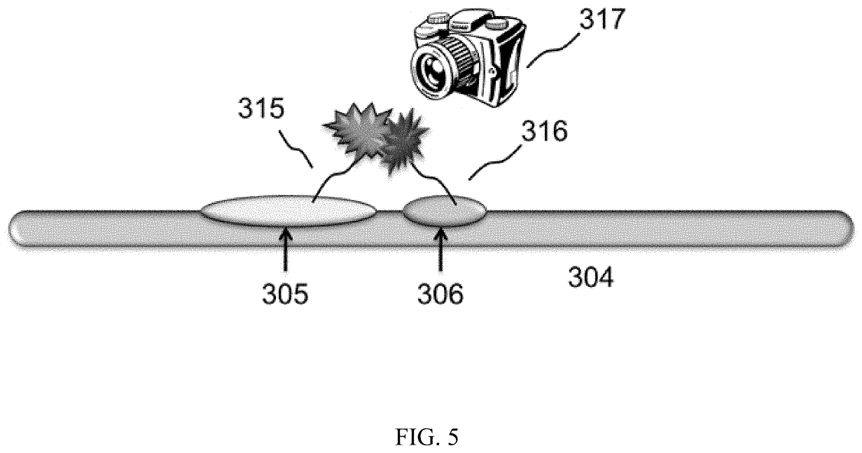

In some aspects, the method further comprises having two independent probes that comprise elements that emit a detectable signal when the two probes are attached to the polynucleotide in sufficiently close proximity. In one embodiment, each probe is labeled with a fluorophore (see, e.g., FIG. 5 (315, 316)). Emission spectra are detected by a detector (317) when the probes are in sufficient proximity to generate a detectable signal. In one embodiment, two probes are labeled with different colored fluorophores. When the probes are in close proximity, the colors will be imaged together (or blended providing a new color) that can be detected with an external sensor, such as a camera or microscope, and evidence that two probes are close in space. In a related embodiment, FRET (or BRET) type detection is used to determine proximity of two probes, such that one fluorescently labeled probe will affect the energy emission spectrum of the other when in close proximity.

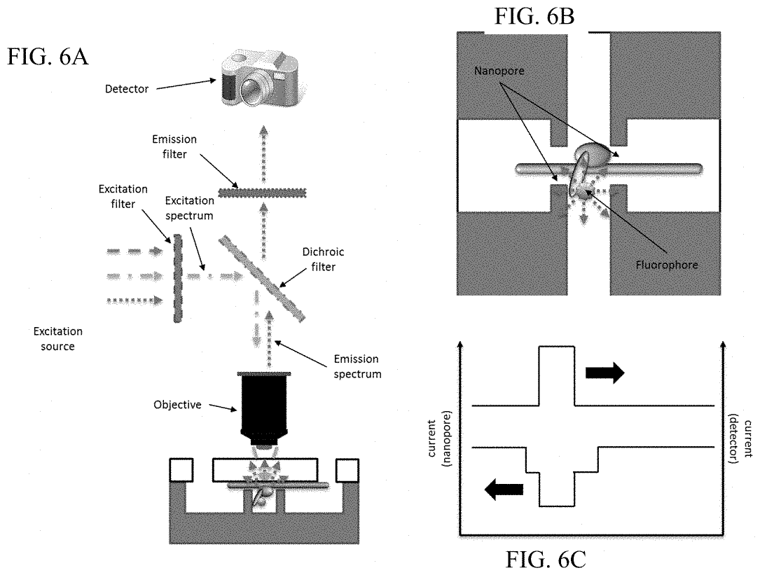

In some embodiments, the detectable label is a fluorophore. To detect the fluorophore, a nanopore device fabricated in-plane with a glass cover can be combined with an epifluorescence microscope to enable dual current amplitude and fluorescence signal detection. FIG. 6A shows how such a device can be used to detect an added fluorophore label. The nanopore device is placed underneath the objective of an epifluorescence microscope. As the nanopore measurement is performed, the microscope is continuously imaging the nanopore region. The nanopore region is illuminated by means of a broadband excitation source that is filtered such that only the wavelengths corresponding to the excitation spectrum of the fluorophore are allowed to pass through. A dichroic filter selectively allows transmission of the wavelengths corresponding to the emission spectrum of the fluorophore while reflecting all other wavelengths. As the fluorophore modified binding molecule passes through the nanopores the fluorophore absorbs the excitation spectrum and reemits an emission spectrum. An emission filter in front of the detector ensure that only wavelengths corresponding to the emission spectrum of the fluorophore are detected. Thus the detector will only have a signal when the fluorophore passes through the nanopore. FIG. 6B shows a top down view of the nanopore device as viewed by the microscope during emission of the fluorophore. FIG. 6C demonstrates how the detection of the fluorophore can be used in conjunction with the signal from the nanopore. The use of two signals enhances the confidence in the detection of the biomolecule.

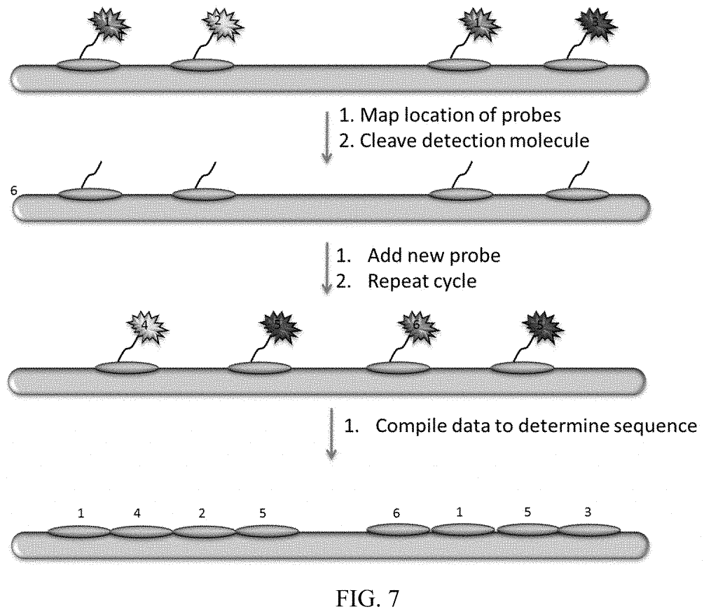

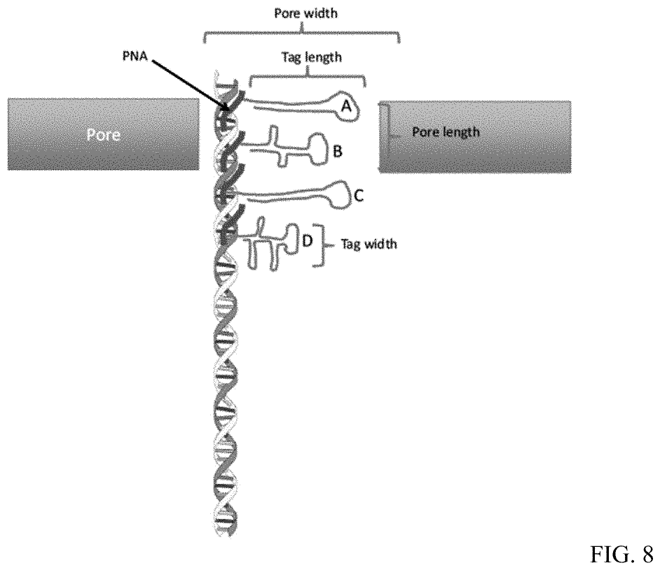

In some aspects, the method further comprises using probes that have feature attached that allow detection by a sensor, but they are attached to the probe using a cleavable linker. Thus a set of probes that can be distinguished from each other in the nanopore are bound to a target bearing polynucleotide. Once that set of probes is detected in the nanopore, the features are cleaved off and a new set of probes are added that also have cleavable detection feature (FIG. 7). The add/cleave/wash cycle can be continued until all sequence information is extracted from a captured target molecule. Example of molecules that aid in probe detection are discussed above. Examples of cleavable linkers are reductant cleavable linkers (disulfide linkers cleaved by TCEP), acid cleavable linker (hydrazone/hydrazide bonds), amino acid sequences that are cleaved by proteases, nucleic acid linkers that are cleaved by endonucleases (sites specific restriction enzymes), base cleavable linkers, or light cleavable linkers [Leriche, Geoffray, Louise Chisholm, and Alain Wagner. “Cleavable linkers in chemical biology.” Bioorganic & medicinal chemistry 20, no. 2 (2012): 571-582.]

Target Motifs

For nucleic acids and polypeptides to which the target sequence detection method is applied, a target binding motif can be a nucleotide or peptide sequence that is recognizable by the probe molecule. Target motifs may be chemically modified (e.g. methylated) or occupied by other molecules (e.g. activator or repressors), and depending on the nature of the probe, the status of the target motif can be elucidated. In some aspects, the target sequence comprises a chemical modification for binding the probe to the polynucleotide. In some aspects, the chemical modification is selected from the group consisting of acetylation, methylation, summolation, glycosylation, phosphorylation, biotinylation, and oxidation.

Probe Molecules

In the present technology, a probe molecule is detected or quantitated by virtue of its binding to the target-bearing polynucleotide.

Probes as used herein are understood to be capable of specifically binding to a site on a polynucleotide, wherein the site is characterized by the sequence or structure. Examples of probe molecules include a PNA (protein nucleic acid), bis-PNA, gamma-PNA, a PNA-conjugate that increases size or charge of PNA. Other examples of probe molecules are from the group consisting of a natural or recombinant protein, protein fusion, DNA binding domain of a protein, peptide, a nucleic acid, oligo nucleotide, TALEN, CRISPR, a PNA (protein nucleic acid), bis-PNA, gamma-PNA, a PNA-conjugate that increases size, charge, fluorescence, or functionality (e.g. oligo labeled), or any other PNA derivatized polymer, and a chemical compound.

In some aspects, the probe comprises a γ-PNA. γ-PNA has a simple modification in a peptide-like backbone, specifically at the γ-position of the N-(2-aminoethyl)glycine backbone, thus generating a chiral center (Rapireddy S., et al., 2007. J. Am. Chem. Soc., 129:15596-600; He G, et al., 2009, J. Am. Chem. Soc., 131:12088-90; Chema V, et al., 2008, Chembiochem 9:2388-91; Dragulescu-Andrasi, A., et al., 2006, J. Am. Chem. Soc., 128:10258-10267). Unlike bis-PNA, γ-PNA can bind to dsDNA without sequence limitation, leaving one of the two DNA strands accessible for further hybridization.

In some aspects, the function of the probe is to hybridize to a polynucleotide with a target sequence by complement base pairing to form a stable complex. The PNA molecule may additionally be bound to additional molecules to form a complex has sufficiently large cross-section surface area to produce a detectable change or contrast in signal amplitude over that of the background, which is the mean or average signal amplitude corresponding to sections of non-probe-bound polynucleotide.

The stability of the binding of the polynucleotide target sequence to the PNA molecule is important in order for it to be detected by a nanopore device. The binding stability must be maintained throughout the period that the target-bearing polynucleotide is being translocated through the nanopore. If the stability is weak, or unstable, the probe can separate from the target polynucleotide and will not be detected as the target-bearing polynucleotide threads through the nanopores.

In a particular embodiment, an example of a probe is a PNA-conjugate in which the PNA portion specifically recognizes a nucleotide sequence and the conjugate portion increases the size/shape/charge differences between different PNA-conjugates.

As illustrated in FIG. 8, ligands A, B, C and D each specifically binds to a site on a DNA molecule, and these ligands can be identified and distinguished from each other by their width, length, size and/or charge. If their corresponding sites are denoted as A, B, C and D, respectively, then identification of the ligands leads to revelation of those DNA sequences, A-B-C-D, in terms of the composition of the sites and order.

Different reactive moieties may be incorporated into the ligands to provide chemical handle to which labels maybe conjugated. Examples of reactive moieties include, but are not limited to, primary amines, carboxylic acids, ketones, amides, aldehydes, boronic acids, hydrazones, thiols, maleimides, alcohols, and hydroxyl groups, and biotin.

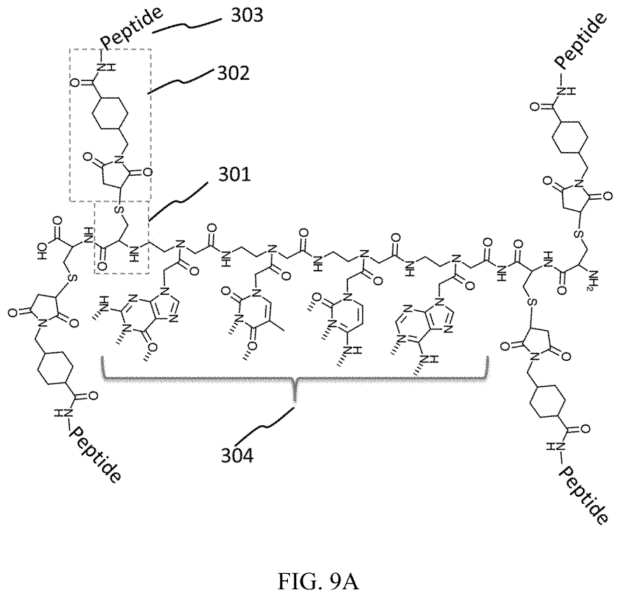

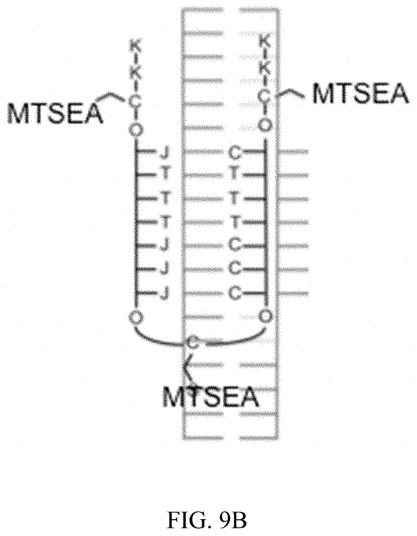

FIG. 9A shows a PNA ligand that has been modified as to increase ligand charge, and therefore facilitate detection by a nanopore. Specifically, this ligand, which binds to the target DNA sequence by complementary base pairing and Hoogsteen base pairing between the bases on the PNA molecule and the bases in the target DNA, has cysteine residues incorporated into the backbone, which provide a free thiol chemical handle for labeling. Here, the cysteine is labeled to a peptide 2-aminoethylmethanethiosulfonate (MTSEA) through a maleimide linker, which provides a means to detect whether the ligand is bound to its target sequence since the label/peptide gives an increase to the ligand charge. This greater charge results in a greater change in current flow through the pore compared to an unlabeled PNA.

In some aspects, to increase the contrast in the change between the ligand bound polynucleotide and other background molecules present in the sample, modification can be made to the pseudo-peptide backbone to change the overall size of the ligand (e.g., PNA) to increase the contrast. See, e.g., FIG. 9B, which shows a PNA that has cysteine residues (301) incorporated that are modified with an SMCC linker (302) to enable conjugation to peptides (303) through the N-terminal amine of the peptide. In addition to adding charge via labeling the ligand (e.g., as in FIG. 9A) selection of more charged amino acids instead of non-polar amino acids can serve to increase the charge of PNA. In addition, small particle, molecules, protein, peptides, or polymers (e.g. PEG) can be conjugated to the pseudo-peptide backbone to enhance the bulk or cross-sectional surface area of the ligand and target-bearing polynucleotide complex. Enhanced bulk serves to improve the signal amplitude contrast so that any differential signal resulting from the increased bulk can be easily detected. Examples of small particle, molecules, protein, or peptides can be conjugated to the pseudo-peptide backbone include but are not limited to alpha-helical forming peptides, nanometer-sized gold particles or rods (e.g. 3 nm), quantum dots, polyethylene glycol (PEG). Method of conjugation of molecules are well known in the art, e.g. in U.S. Pat. Nos. 5,180,816, 6,423,685, 6,706,252, 6,884,780, and 7,022,673, which are hereby incorporated by reference in their entirety.

The embodiments above describe PEG labeling through cysteine residues, however other residues can also be used. For example, Lysine residue are easily interchanged with cysteine residues to enable linkage chemistry using NHS-esters and free amines. Also, PEG can easily be interchanged with other bulk-adding constituents, like Dendrons, beads, or rods. between the bifunctional linker and the PNA, or to directly couple the Dendron. Someone skilled in the art would recognize the flexibility of this system in that the amino acid can be changed and linkage chemistry modified for that particular amino acid, e.g. Serine reactive isocyanates. Some examples of linkage chemistry that can be used for this reaction is listed in the table below.

TABLE 1
Linkage Chemistry

| Reactive Group | Target Functional Group |
|---|---|
| aryl azide | nonselective or primary amine |
| carbodiimide | amine/carboxyl |
| hydrazide | carbohydrate |
| hydroxymethyl phosphine | amine |
| imidoester | amine |
| isocyanate | hydroxyl |
| carbonyl | hydrazine |
| maleimide | sulfhydryl |
| NHS-ester | amine |
| PFP-ester | amine |
| psoralen | thymine |
| pyridyl disulfide | sulfhydryl |
| vinyl sulfone | sulfhydryl amine, hydroxyl |

FIGS. 3A, 3B, 4, 5, 9A, 9B and 18A show PNA probes that have been modified as to increase probe size, contain an epitope, contain ssDNA oligomers, contain fluorophores, additional charge, or additional size to facilitate detection or to detect that two probes are in close proximity.

Different reactive moieties can be incorporated into the probes to provide a chemical handle to which labels maybe conjugated. Examples of reactive moieties include, but are not limited to, primary amines, carboxylic acids, ketones, amides, aldehydes, boronic acids, hydrazones, thiols, maleimides, alcohols, and hydroxyl groups, and biotin.

A common method for incorporating the chemical handles are to include a specific amino acid into the backbone of the probe. Examples include, but are not limited to, cysteines (provide thiolates), lysines (provides free amines), threonine (provides hydroxyl), glutamate and aspartate (provides carboxylic acids).

Different types of labels can be added using the reactive moieties. These includes labels that:
1. increase the size of the probe, e.g. biotin/streptavidin, peptide, nucleic acid
2. change the charge of the probe, e.g. a charged peptide (6×HIS (SEQ ID NO: 2)), or protein (e.g., charybdotoxin), or small molecule or peptide (e.g. MTSET).
3. change or add fluorescence to the probe, e.g. common fluorophores, FITC, Rhodamine, Cy3, Cy5.
4. Provide an epitope or interaction site for binding a third molecule, e.g. peptides for binding antibody

Multiplexing

In some embodiments, rather than including probes of the same kind, as described above, a collection of different probes are added that each bind to a unique site or target motif.

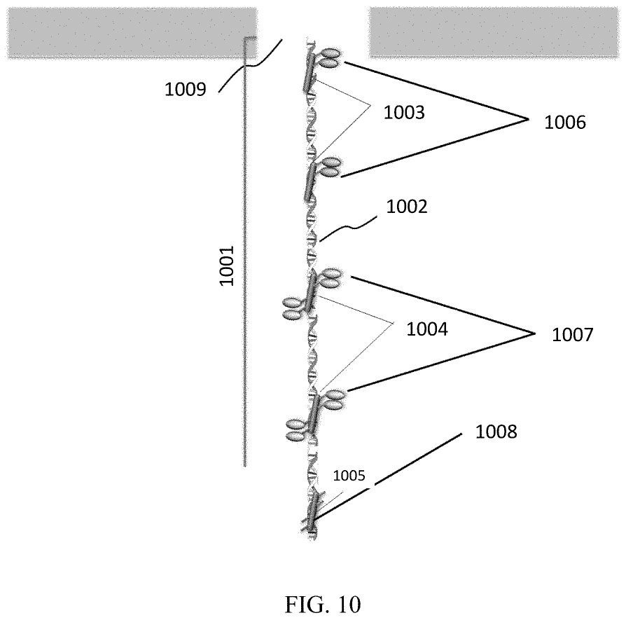

With such a setting, multiple different probes can be used to detect multiple different target sites within the same target bearing polynucleotide. FIG. 10 illustrates such a method. Here, a double-stranded DNA 1002 contains multiple different target motifs, two copies of 1003, two copies of 1004, and one copy of 1005.

By using probes that each provide a unique current profile (e.g., by differing in size) 1006, 1007, and 1008, the present technology can detect different target motifs within the same molecule, providing a means for multiplexing target motif detection. Further, by enumerating how many of each unique probes are bound, number of each target (or copy number) can be determined. By tuning conditions that impact the bindings, the system can obtain more detailed binding dynamic information.

Similarly, multiplexing can be accomplished by having a collection of probes with differing attributes and mixed-and-matched in any number of combination, the only requirement is that probes that bind to a different sequence are discernable from each other. For example, an experiment could use probes that are distinguishable by size and additional probes that are distinguishable by size (FIGS. 9A, 9B and 19).

An additional method of multiplexing involves designing probes that bind to the polynucleotide at known sequences at fixed positions from each other to interrogate a sample that contains a collection of nucleic acids from different species. As an example of this method, if we are testing a water source for three different bacteria of know sequence, we can position two probes 1000 base pairs apart for species A, 3000 bp apart for species B, and 5000 base pairs apart for species C. If probes are detected that are 1000 base pairs and 3000 base pairs apart, then species A and B are present, but not C to a detectable degree. This same method of designed spacing can also be used to multiplex detection of known or mutated sequence in a particular target sample.

Nanopore Devices

A nanopore device, as provided, includes a pore that forms an opening in a structure separating an interior space of the device into two volumes, and is configured to identify objects (for example, by detecting changes in parameters indicative of objects) passing through the pore, e.g., with a sensor. Nanopore devices used for the methods described herein are also disclosed in PCT Publication WO/2013/012881, incorporated by reference in entirety.

The pore(s) in the nanopore device are of a nano scale or micro scale. In one aspect, each pore has a size that allows a small or large molecule or microorganism to pass. In one aspect, each pore is at least about 1 nm in diameter. Alternatively, each pore is at least about 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, 90 nm, or 100 nm in diameter.

In one aspect, the pore is no more than about 100 nm in diameter. Alternatively, the pore is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm in diameter.

In some aspects, each pore is at least about 100 nm, 200 nm, 500 nm, 1000 nm, 2000 nm, 3000 nm, 5000 nm, 10000 nm, 20000 nm, or 30000 nm in diameter. In one aspect, the pore is no more than about 100000 nm in diameter. Alternatively, the pore is no more than about 50000 nm, 40000 nm, 30000 nm, 20000 nm, 10000 nm, 9000 nm, 8000 nm, 7000 nm, 6000 nm, 5000 nm, 4000 nm, 3000 nm, 2000 nm, or 1000 nm in diameter.

In one aspect, the pore has a diameter that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In some aspects, the pore(s) in the nanopore device are of a larger scale for detecting large microorganisms or cells. In one aspect, each pore has a size that allows a large cell or microorganism to pass. In one aspect, each pore is at least about 100 nm in diameter. Alternatively, each pore is at least about 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, 1600 nm, 1700 nm, 1800 nm, 1900 nm, 2000 nm, 2500 nm, 3000 nm, 3500 nm, 4000 nm, 4500 nm, or 5000 nm in diameter.

In one aspect, the pore is no more than about 100,000 nm in diameter. Alternatively, the pore is no more than about 90,000 nm, 80,000 nm, 70,000 nm, 60,000 nm, 50,000 nm, 40,000 nm, 30,000 nm, 20,000 nm, 10,000 nm, 9000 nm, 8000 nm, 7000 nm, 6000 nm, 5000 nm, 4000 nm, 3000 nm, 2000 nm, or 1000 nm in diameter.

In one aspect, the pore has a diameter that is between about 100 nm and about 10000 nm, or alternatively between about 200 nm and about 9000 nm, or between about 300 nm and about 8000 nm, or between about 400 nm and about 7000 nm, or between about 500 nm and about 6000 nm, or between about 1000 nm and about 5000 nm, or between about 1500 nm and about 3000 nm.

In some aspects, the nanopore device further includes means to move a polymer scaffold across the pore and/or means to identify objects that pass through the pore. Further details are provided below, described in the context of a two-pore device.

Compared to a single-pore nanopore device, a two-pore device can be more easily configured to provide good control of speed and direction of the movement of the polymer scaffold across the pores.

In certain embodiments, the nanopore device includes a plurality of chambers, each chamber in communication with an adjacent chamber through at least one pore. Among these pores, two pores, namely a first pore and a second pore, are placed so as to allow at least a portion of a polymer scaffold to move out of the first pore and into the second pore. Further, the device includes a sensor capable of identifying the polymer scaffold during the movement. In one aspect, the identification entails identifying individual components of the polymer scaffold. In another aspect, the identification entails identifying fusion molecules and/or target analytes bound to the polymer scaffold. When a single sensor is employed, the single sensor may include two electrodes placed at both ends of a pore to measure an ionic current across the pore. In another embodiment, the single sensor comprises a component other than electrodes.

In one aspect, the device includes three chambers connected through two pores. Devices with more than three chambers can be readily designed to include one or more additional chambers on either side of a three-chamber device, or between any two of the three chambers. Likewise, more than two pores can be included in the device to connect the chambers.

In one aspect, there can be two or more pores between two adjacent chambers, to allow multiple polymer scaffolds to move from one chamber to the next simultaneously. Such a multi-pore design can enhance throughput of polymer scaffold analysis in the device.

In some aspects, the device further includes means to move a polymer scaffold from one chamber to another. In one aspect, the movement results in loading the polymer scaffold across both the first pore and the second pore at the same time. In another aspect, the means further enables the movement of the polymer scaffold, through both pores, in the same direction.

For instance, in a three-chamber two-pore device (a “two-pore” device), each of the chambers can contain an electrode for connecting to a power supply so that a separate voltage can be applied across each of the pores between the chambers.

In accordance with an embodiment of the present disclosure, provided is a device comprising an upper chamber, a middle chamber and a lower chamber, wherein the upper chamber is in communication with the middle chamber through a first pore, and the middle chamber is in communication with the lower chamber through a second pore. Such a device may have any of the dimensions or other characteristics previously disclosed in U.S. Publ. No. 2013-0233709, entitled Dual-Pore Device, which is herein incorporated by reference in its entirety.

In some embodiments as shown in FIG. 7A, the device includes an upper chamber 705 (Chamber A), a middle chamber 704 (Chamber B), and a lower chamber 703 (Chamber C). The chambers are separated by two separating layers or membranes (701 and 702) each having a separate pore (711 or 712). Further, each chamber contains an electrode (721, 722 or 723) for connecting to a power supply. The annotation of upper, middle and lower chamber is in relative terms and does not indicate that, for instance, the upper chamber is placed above the middle or lower chamber relative to the ground, or vice versa.

Each of the pores 711 and 712 independently has a size that allows a small or large molecule or microorganism to pass. In one aspect, each pore is at least about 1 nm in diameter. Alternatively, each pore is at least about 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, 90 nm, or 100 nm in diameter.

In one aspect, the pore is no more than about 100 nm in diameter. Alternatively, the pore is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm in diameter.

In one aspect, the pore has a diameter that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In other aspects, each pore is at least about 100 nm, 200 nm, 500 nm, 1000 nm, 2000 nm, 3000 nm, 5000 nm, 10000 nm, 20000 nm, or 30000 nm in diameter. In one aspect, each pore is 50,000 nm to 100,000 nm in diameter. In one aspect, the pore is no more than about 100000 nm in diameter. Alternatively, the pore is no more than about 50000 nm, 40000 nm, 30000 nm, 20000 nm, 10000 nm, 9000 nm, 8000 nm, 7000 nm, 6000 nm, 5000 nm, 4000 nm, 3000 nm, 2000 nm, or 1000 nm in diameter.

In some aspects, the pore has a substantially round shape. “Substantially round”, as used here, refers to a shape that is at least about 80 or 90% in the form of a cylinder. In some embodiments, the pore is square, rectangular, triangular, oval, or hexangular in shape.

Each of the pores 711 and 712 independently has a depth (i.e., a length of the pore extending between two adjacent volumes). In one aspect, each pore has a depth that is least about 0.3 nm. Alternatively, each pore has a depth that is at least about 0.6 nm, 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, or 90 nm.

In one aspect, each pore has a depth that is no more than about 100 nm. Alternatively, the depth is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm.

In one aspect, the pore has a depth that is between about 1 nm and about 100 nm, or alternatively, between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In some aspects, the nanopore extends through a membrane. For example, the pore may be a protein channel inserted in a lipid bilayer membrane or it may be engineered by drilling, etching, or otherwise forming the pore through a solid-state substrate such as silicon dioxide, silicon nitride, grapheme, or layers formed of combinations of these or other materials. In some aspects, the length or depth of the nanopore is sufficiently large so as to form a channel connecting two otherwise separate volumes. In some such aspects, the depth of each pore is greater than 100 nm, 200 nm, 300 nm, 400 nm, 500 nm, 600 nm, 700 nm, 800 nm, or 900 nm. In some aspects, the depth of each pore is no more than 2000 nm or 1000 nm.

In one aspect, the pores are spaced apart at a distance that is between about 10 nm and about 1000 nm. In some aspects, the distance between the pores is greater than 1000 nm, 2000 nm, 3000 nm, 4000 nm, 5000 nm, 6000 nm, 7000 nm, 8000 nm, or 9000 nm. In some aspects, the pores are spaced no more than 30000 nm, 20000 nm, or 10000 nm apart. In one aspect, the distance is at least about 10 nm, or alternatively, at least about 20 nm, 30 nm, 40 nm, 50 nm, 60 nm, 70 nm, 80 nm, 90 nm, 100 nm, 150 nm, 200 nm, 250 nm, or 300 nm. In another aspect, the distance is no more than about 1000 nm, 900 nm, 800 nm, 700 nm, 600 nm, 500 nm, 400 nm, 300 nm, 250 nm, 200 nm, 150 nm, or 100 nm.

In yet another aspect, the distance between the pores is between about 20 nm and about 800 nm, between about 30 nm and about 700 nm, between about 40 nm and about 500 nm, or between about 50 nm and about 300 nm.

The two pores can be arranged in any position so long as they allow fluid communication between the chambers and have the prescribed size and distance between them. In one aspect, the pores are placed so that there is no direct blockage between them. Still, in one aspect, the pores are substantially coaxial, as illustrated in FIG. 7A.

In one aspect, as shown in FIG. 7A, the device, through the electrodes 721, 722, and 723 in the chambers 703, 704, and 705, respectively, is connected to one or more power supplies. In some aspects, the power supply includes a voltage-clamp or a patch-clamp, which can supply a voltage across each pore and measure the current through each pore independently. In this respect, the power supply and the electrode configuration can set the middle chamber to a common ground for both power supplies. In one aspect, the power supply or supplies are configured to apply a first voltage V₁ between the upper chamber 705 (Chamber A) and the middle chamber 704 (Chamber B), and a second voltage V₂ between the middle chamber 704 and the lower chamber 703 (Chamber C).

In some aspects, the first voltage V₁ and the second voltage V₂ are independently adjustable. In one aspect, the middle chamber is adjusted to be a ground relative to the two voltages. In one aspect, the middle chamber comprises a medium for providing conductance between each of the pores and the electrode in the middle chamber. In one aspect, the middle chamber includes a medium for providing a resistance between each of the pores and the electrode in the middle chamber. Keeping such a resistance sufficiently small relative to the nanopore resistances is useful for decoupling the two voltages and currents across the pores, which is helpful for the independent adjustment of the voltages.

Adjustment of the voltages can be used to control the movement of charged particles in the chambers. For instance, when both voltages are set in the same polarity, a properly charged particle can be moved from the upper chamber to the middle chamber and to the lower chamber, or the other way around, sequentially. In some aspects, when the two voltages are set to opposite polarity, a charged particle can be moved from either the upper or the lower chamber to the middle chamber and kept there.

The adjustment of the voltages in the device can be particularly useful for controlling the movement of a large molecule, such as a charged polymer scaffold, that is long enough to cross both pores at the same time. In such an aspect, the direction and the speed of the movement of the molecule can be controlled by the relative magnitude and polarity of the voltages as described below.

The device can contain materials suitable for holding liquid samples, in particular, biological samples, and/or materials suitable for nanofabrication. In one aspect, such materials include dielectric materials such as, but not limited to, silicon, silicon nitride, silicon dioxide, graphene, carbon nanotubes, TiO₂, HfO₂, Al₂O₃, or other metallic layers, or any combination of these materials. In some aspects, for example, a single sheet of graphene membrane of about 0.3 nm thick can be used as the pore-bearing membrane.

Devices that are microfluidic and that house two-pore microfluidic chip implementations can be made by a variety of means and methods. For a microfluidic chip comprised of two parallel membranes, both membranes can be simultaneously drilled by a single beam to form two concentric pores, though using different beams on each side of the membranes is also possible in concert with any suitable alignment technique. In general terms, the housing ensures sealed separation of Chambers A-C. In one aspect as shown in FIG. 7B, the housing would provide minimal access resistance between the voltage electrodes 721, 722, and 723 and the nanopores 711 and 712, to ensure that each voltage is applied principally across each pore.

In one aspect, the device includes a microfluidic chip (labeled as “Dual-core chip”) is comprised of two parallel membranes connected by spacers. Each membrane contains a pore drilled by a single beam through the center of the membrane. Further, the device preferably has a Teflon® housing for the chip. The housing ensures sealed separation of Chambers A-C and provides minimal access resistance for the electrode to ensure that each voltage is applied principally across each pore.

More specifically, the pore-bearing membranes can be made with transmission electron microscopy (TEM) grids with a 5-100 nm thick silicon, silicon nitride, or silicon dioxide windows. Spacers can be used to separate the membranes, using an insulator, such as SU-8, photoresist, PECVD oxide, ALD oxide, ALD alumina, or an evaporated metal material, such as Ag, Au, or Pt, and occupying a small volume within the otherwise aqueous portion of Chamber B between the membranes. A holder is seated in an aqueous bath that is comprised of the largest volumetric fraction of Chamber B. Chambers A and C are accessible by larger diameter channels (for low access resistance) that lead to the membrane seals.

A focused electron or ion beam can be used to drill pores through the membranes, naturally aligning them. The pores can also be sculpted (shrunk) to smaller sizes by applying a correct beam focusing to each layer. Any single nanopore drilling method can also be used to drill the pair of pores in the two membranes, with consideration to the drill depth possible for a given method and the thickness of the membranes. Predrilling a micro-pore to a prescribed depth and then a nanopore through the remainder of the membranes is also possible to further refine the membrane thickness.

In another aspect, the insertion of biological nanopores into solid-state nanopores to form a hybrid pore can be used in either or both pores in the two-pore method. The biological pore can increase the sensitivity of the ionic current measurements, and is useful when only single-stranded polynucleotides are to be captured and controlled in the two-pore device, e.g., for sequencing.

By virtue of the voltages present at the pores of the device, charged molecules can be moved through the pores between chambers. Speed and direction of the movement can be controlled by the magnitude and polarity of the voltages. Further, because each of the two voltages can be independently adjusted, the direction and speed of the movement of a charged molecule can be finely controlled in each chamber.

One example concerns a charged polymer scaffold, such as a DNA, having a length that is longer than the combined distance that includes the depth of both pores plus the distance between the two pores. For example, a 1000 by dsDNA is about 340 nm in length, and would be substantially longer than the 40 nm spanned by two 10 nm-deep pores separated by 20 nm. In a first step, the polynucleotide is loaded into either the upper or the lower chamber. By virtue of its negative charge under a physiological condition at a pH of about 7.4, the polynucleotide can be moved across a pore on which a voltage is applied. Therefore, in a second step, two voltages, in the same polarity and at the same or similar magnitudes, are applied to the pores to move the polynucleotide across both pores sequentially.

At about the time when the polynucleotide reaches the second pore, one or both of the voltages can be changed. Since the distance between the two pores is selected to be shorter than the length of the polynucleotide, when the polynucleotide reaches the second pore, it is also in the first pore. A prompt change of polarity of the voltage at the first pore, therefore, will generate a force that pulls the polynucleotide away from the second pore as illustrated in FIG. 7C.

Assuming that the two pores have identical voltage-force influence and |V₁|=|V₂|+δV, the value δV>0 (or <0) can be adjusted for tunable motion in the V₁| (or V₂) direction. In practice, although the voltage-induced force at each pore will not be identical with V₁=V₂, calibration experiments can identify the appropriate bias voltage that will result in equal pulling forces for a given two-pore chip; and variations around that bias voltage can then be used for directional control.

If, at this point, the magnitude of the voltage-induced force at the first pore is less than that of the voltage-induced force at the second pore, then the polynucleotide will continue crossing both pores towards the second pore, but at a lower speed. In this respect, it is readily appreciated that the speed and direction of the movement of the polynucleotide can be controlled by the polarities and magnitudes of both voltages. As will be further described below, such a fine control of movement has broad applications.

Accordingly, in one aspect, provided is a method for controlling the movement of a charged polymer scaffold through a nanopore device. The method entails (a) loading a sample comprising a charged polymer scaffold in one of the upper chamber, middle chamber or lower chamber of the device of any of the above embodiments, wherein the device is connected to one or more power supplies for providing a first voltage between the upper chamber and the middle chamber, and a second voltage between the middle chamber and the lower chamber; (b) setting an initial first voltage and an initial second voltage so that the polymer scaffold moves between the chambers, thereby locating the polymer scaffold across both the first and second pores; and (c) adjusting the first voltage and the second voltage so that both voltages generate force to pull the charged polymer scaffold away from the middle chamber (voltage-competition mode), wherein the two voltages are different in magnitude, under controlled conditions, so that the charged polymer scaffold moves across both pores in either direction and in a controlled manner.

To establish the voltage-competition mode in step (c), the relative force exerted by each voltage at each pore is to be determined for each two-pore device used, and this can be done with calibration experiments by observing the influence of different voltage values on the motion of the polynucleotide, which can be measured by sensing known-location and detectable features in the polynucleotide, with examples of such features detailed later in this disclosure. If the forces are equivalent at each common voltage, for example, then using the same voltage value at each pore (with common polarity in upper and lower chambers relative to grounded middle chamber) creates a zero net motion in the absence of thermal agitation (the presence and influence of Brownian motion is discussed below). If the forces are not equivalent at each common voltage, achieving equal forces involves the identification and use of a larger voltage at the pore that experiences a weaker force at the common voltage. Calibration for voltage-competition mode can be done for each two-pore device, and for specific charged polymers or molecules whose features influence the force when passing through each pore.

In one aspect, the sample containing the charged polymer scaffold is loaded into the upper chamber and the initial first voltage is set to pull the charged polymer scaffold from the upper chamber to the middle chamber and the initial second voltage is set to pull the polymer scaffold from the middle chamber to the lower chamber. Likewise, the sample can be initially loaded into the lower chamber, and the charged polymer scaffold can be pulled to the middle and the upper chambers.

In another aspect, the sample containing the charged polymer scaffold is loaded into the middle chamber; the initial first voltage is set to pull the charged polymer scaffold from the middle chamber to the upper chamber; and the initial second voltage is set to pull the charged polymer scaffold from the middle chamber to the lower chamber.

In one aspect, the adjusted first voltage and second voltage at step (c) are about 10 times to about 10,000 times as high, in magnitude, as the difference/differential between the two voltages. For instance, the two voltages can be 90 mV and 100 mV, respectively. The magnitude of the two voltages, about 100 mV, is about 10 times of the difference/differential between them, 10 mV. In some aspects, the magnitude of the voltages is at least about 15 times, 20 times, 25 times, 30 times, 35 times, 40 times, 50 times, 100 times, 150 times, 200 times, 250 times, 300 times, 400 times, 500 times, 1000 times, 2000 times, 3000 times, 4000 times, 5000 times, 6000 times, 7000 times, 8000 times or 9000 times as high as the difference/differential between them. In some aspects, the magnitude of the voltages is no more than about 10000 times, 9000 times, 8000 times, 7000 times, 6000 times, 5000 times, 4000 times, 3000 times, 2000 times, 1000 times, 500 times, 400 times, 300 times, 200 times, or 100 times as high as the difference/differential between them.

In one aspect, real-time or on-line adjustments to the first voltage and the second voltage at step (c) are performed by active control or feedback control using dedicated hardware and software, at clock rates up to hundreds of megahertz. Automated control of the first or second or both voltages is based on feedback of the first or second or both ionic current measurements.

Sensors

In certain embodiments, the nanopore devices of the present invention include one or more sensors to carry out the identification of the binding status of the target motifs.

The sensors used in the device can be any sensor suitable for identifying a molecule or particle, such as a charged polymer. For instance, a sensor can be configured to identify the charged polymer by measuring a current, a voltage, pH, an optical feature or residence time associated with the charged polymer or one or more individual components of the charged polymer. In some embodiments, the sensor includes a pair of electrodes placed at opposing sides of a pore to measure an ionic current through the pore when a molecule or particle, in particular a charged polymer (e.g., a polynucleotide), moves through the pore.

In certain embodiments, the sensor measures an optical feature of the polymer or a component (or unit) of the polymer. One example of such measurement includes identification by infrared (or ultraviolet) spectroscopy of an absorption band unique to a particular unit.

When residence time measurements are used, they will correlate the size of the unit to the specific unit based on the length of time it takes to pass through the sensing device.

In some embodiments, the sensor is functionalized with reagents that form distinct non-covalent bonds with each of the probes. In this respect, the gap can be larger and still allow effective measuring. For instance, a 5 nm gap can be used to detect a probe/target complex measuring roughly 5 nm. Tunnel sensing with a functionalized sensor is termed “recognition tunneling.” Using a Scanning Tunneling Microscope (STM) with recognition tunneling, a probe bound to a target motif is easily identified.

Therefore, the methods of the present technology can provide charged polynucleotide (e.g., DNA) delivery rate control for one or more recognition tunneling sites, each positioned in one or both of the nanopore channels or between the pores, and voltage control can ensure that each probe/target complex resides in each site for a sufficient duration for robust identification.

Sensors in the devices and methods of the present disclosure can comprise gold, platinum, graphene, or carbon, or other suitable materials. In a particular aspect, the sensor includes parts made of graphene. Graphene can act as a conductor and an insulator, thus tunneling currents through the graphene and across the nanopore can sequence the translocating DNA.

In some embodiments, the tunnel gap has a width that is from about 1 nm to about 20 nm. In one aspect, the width of the gap is at least about 1 nm, or alternatively at least about 1.5, 2, 2.5, 3, 3.5, 4, 4.5, 5, 6, 7, 8, 9, 10, 12 or 15 nm. In another aspect, the width of the gap is not greater than about 20 nm, or alternatively not greater than about 19, 18, 17, 16, 15, 14, 13, 12, 11, 10, 9, 8, 7, 6, 5, 4, 3, or 2 nm. In some aspects, the width is between about 1 nm and about 15 nm, between about 1 nm and about 10 nm, between about 2 nm and about 10 nm, between about 2.5 nm and about 10 nm, or between about 2.5 nm and about 5 nm.

In some embodiments, the sensor is an electric sensor. In some embodiments, the sensor detects a fluorescent detection means when the probe has is label to create unique fluorescent signature. A radiation source at the outlet can be used to detect that signature.

It is to be understood that while the invention has been described in conjunction with the above embodiments, that the foregoing description and following examples are intended to illustrate and not limit the scope of the invention. Other aspects, advantages and modifications within the scope of the invention will be apparent to those skilled in the art to which the invention pertains.

### EXAMPLES

### Example 1—DNA Alone in a Solid-State Nanopore Experiment

Nanopore instruments use a sensitive voltage-clamp amplifier to apply a voltage V across the pore while measuring the ionic current I₀ through the open pore. When a single charged molecule such as a double-stranded DNA (dsDNA) is captured and driven through the pore by electrophoresis, the measured current shifts from I₀ to I_{B}, and the shift amount ΔI=I₀−I_{B} and duration t_{D} are used to characterize the event. After recording many events during an experiment, distributions of the events on a ΔI vs. t_{D} plot are analyzed to characterize the corresponding molecule in a population on the plot. In this way, nanopores provide a simple, label-free, purely electrical single-molecule method for biomolecular sensing.

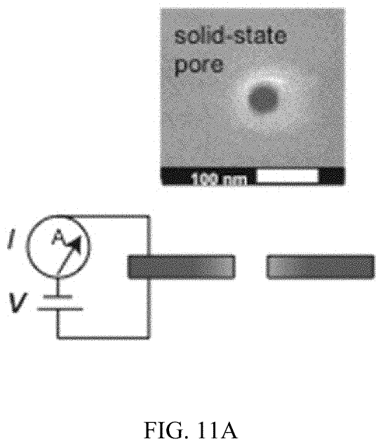

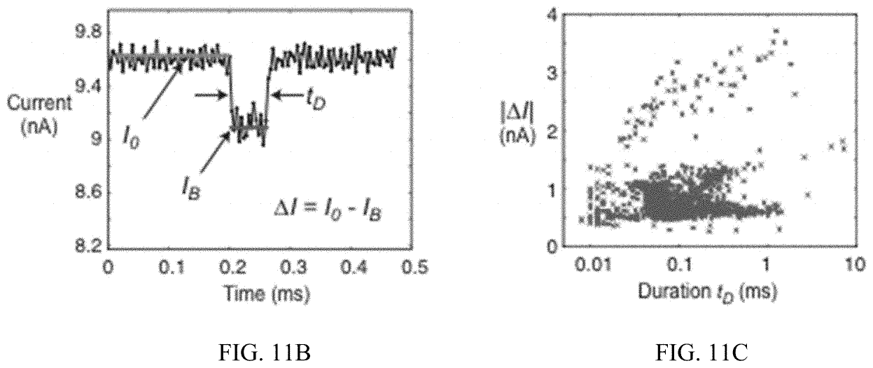

A single nanopore fabricated in silicon nitride (SiN) substrate is a 40 nm diameter pore in 100 nm thick SiN membrane (FIG. 11A) is shown as an example of a solid-state nanopore. In FIG. 11B, the representative current trace shows a blockade event caused by a 5.6 kb dsDNA passing in a single file manner (unfolded) through an 11 nm diameter nanopore in 10 nm thick SiN at 200 mV in buffer containing 1M KCl. The current is attenuated when DNA passes through the pore for KCl concentrations at or above 0.3 M, whereas the current is enhanced when DNA passes through a pore for KCl concentrations below 0.3 M. The mean open channel current is I₀=9.6 nA, with mean event amplitude I_{B}=9.1 nA, and mean event duration t_{D}=0.064 ms. The amplitude shift from the translocation of a dsDNA molecule through the nanopore is ΔI=I₀−I_{B}=0.5 nA. In FIG. 11C, the scatter plot shows vs. t_{D} for all 1301 events recorded over 16 minutes.

### Example 2—Capture Molecules Comprising PNA and Biotin for Target Sequence Detection

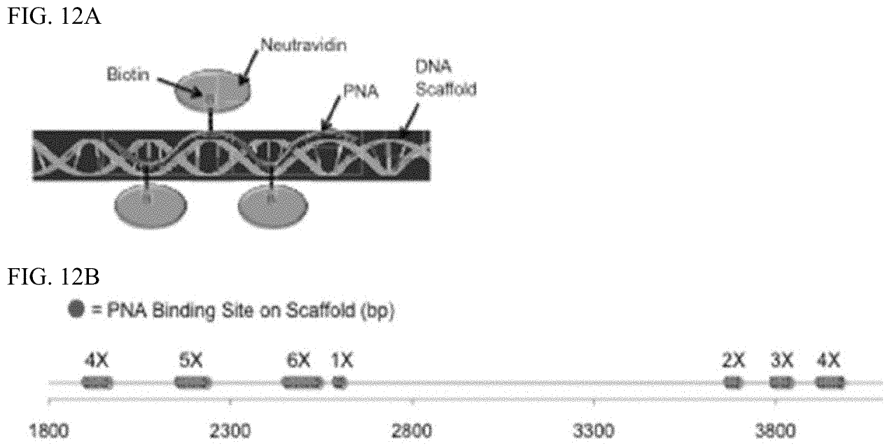

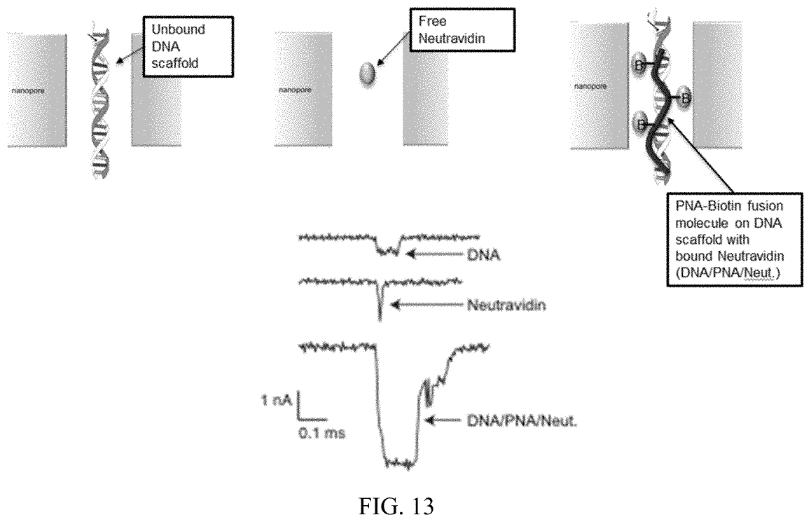

We have demonstrated an approach that permits binding detection of a compound in solution by an engineered polymer scaffold. We provide a PNA probe that has been modified to contain a biotin moiety that binds neutravidin. Neutravidin increases bulk and therefore makes the PNA detectable in nanopores that are large (e.g., 15-30 nm diameter). In particular, we engineered a 5.6 kb dsDNA scaffold to bind to a 12-mer peptide-nucleic-acid (PNA) probe molecule, with each PNA probe having 3 biotinylated sites each for binding a neutravidin (FIG. 12A). We engineered the dsDNA scaffold to have 25 distinct sites (binding motifs) that bind to our PNA probe (FIG. 12B). We provided a solution comprising either the polymer scaffold only, free neutravidin, or probe/DNA complex (FIG. 13). The resulting current event signatures from each population (FIG. 13) show that the DNA/PNA/Neutravidin complexes cause translocation current signatures that are detectable above other background event types (e.g., unbound DNA alone, Neutravidin alone, PNA/Neutravidin alone) and can therefore be identified in the nanopore device. In the remainder of this example, we show that DNA/PNA/Neutravidin complexes can be detected with a nanopore with high confidence.

The probe that binds the specific DNA sequence is a protein nucleic acid molecule (PNA) that binds to the unique sequence (GAAAGTGAAAGT (SEQ ID NO: 1), uSeq1) that is repeated 25 times throughout the scaffold. The PNA used in the experiment had the sequence GAA*AGT*GAA*AGT (SEQ ID NO: 1) where the * indicates that a biotin was incorporated into the PNA backbone at the gamma position by coupling to a lysine amino acid, and thus, each PNA has three biotin molecules and potentially binds 3 neutravidin molecules (PNABio). To bind the PNA, a 60 nM scaffold is heated to 95° C. for 2 minutes, cooled to 60° C. and incubated with a 10× excess of PNA to possible PNA-binding sites on the scaffold in 15 mM NaCl for 1 hr and then cooled to 4° C. The excess PNA is dialyzed out (20 k MWCO, Thermo Scientific) for 2 hrs against 10 mM Tris pH 8.0. This DNA/PNA complex is then labeled with a 10-fold excess Neutravidin protein (Pierce/Thermo Scientific) to possible biotin sites bound to the scaffolds (assuming a 60% reduction of PNA during dialysis). The reaction is electrophoresed as described above to assess purity, concentration, and potential aggregation.

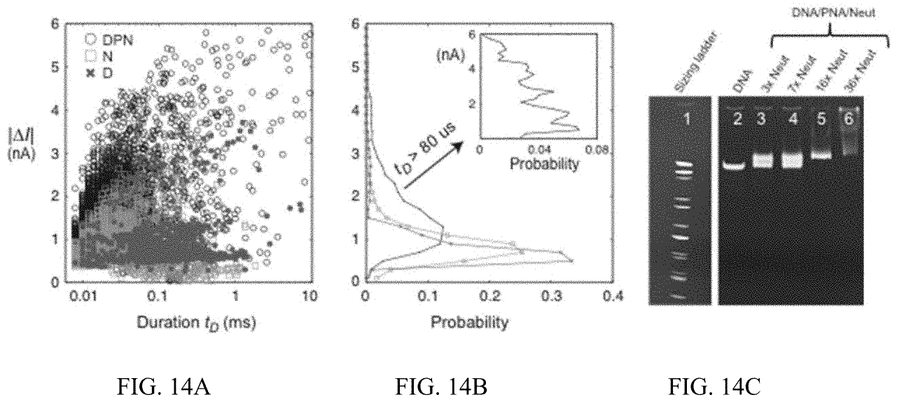

FIGS. 14A-B show data comparing ΔI vs. t_{D} distributions from three separate experiments: DNA alone (D), Neutravidin alone (N), and D/P/N reagents (DPN). The largest |ΔI| events in the D/P/N experiment are attributed to D/P/N complexes (FIG. 13), providing a simple criteria for tagging events based on their binding state (i.e., unbound, scaffold with PNA, and scaffold with PNA and Neutravidin bound). Specifically, we can flag an event as corresponding to a D/P/N complex if |ΔI|>4 nA for that event. For the data sets in FIG. 14A, 9.3% (390) of events in the D/P/N experiment have |ΔI|>4 nA, with only 0.46% of D and 0.16% of N events in controls exceeding 4 nA. In a separate experiment (data not shown) with a 7 nm diameter pore at 1M KCl and 200 mV applied, in a control with only PNA and Neutravidin at 0.4 nM concentration, no events (0%) exceeded 4 nA. Applying our mathematical criteria, the random variable Q={Fraction of flagged events} has a binomial distribution, and using this and other statistical modeling tools, we can compute the 99% confidence interval for this data set as Q=9.29±1.15%. Since 9.29%>0.46% (the max false-positive %) is satisfied well within the 99% confidence interval for Q, we have a positive test result, and in under 8 minutes of data gathering. In fact the same 99% confidence is achieved for this data set with only the first 60 seconds of the data. The gel shift (FIG. 14C) shows that scaffold DNA migration is retarded in a Neutravidin dependent manner; this guided us to using the 10× concentration in this preliminary experiment, as it appeared all DNA is labeled and a nearly homogenous population is created

### Example 3—Vspr Protein Binding to DNA Scaffold and Nanopore Detection

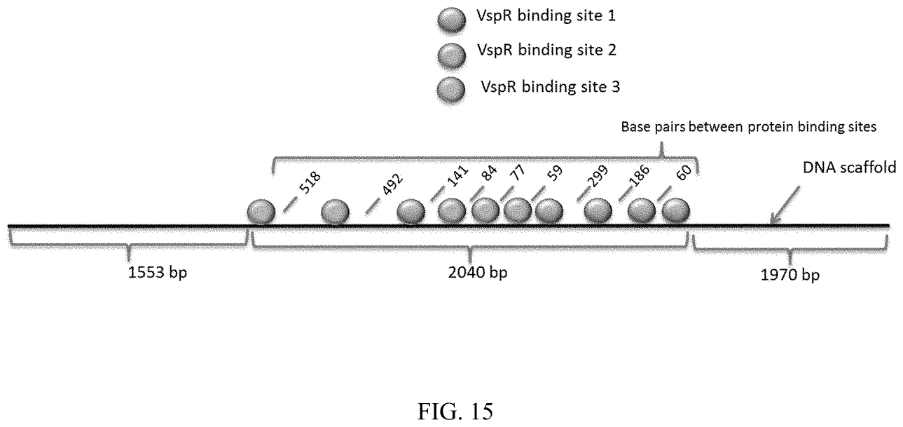

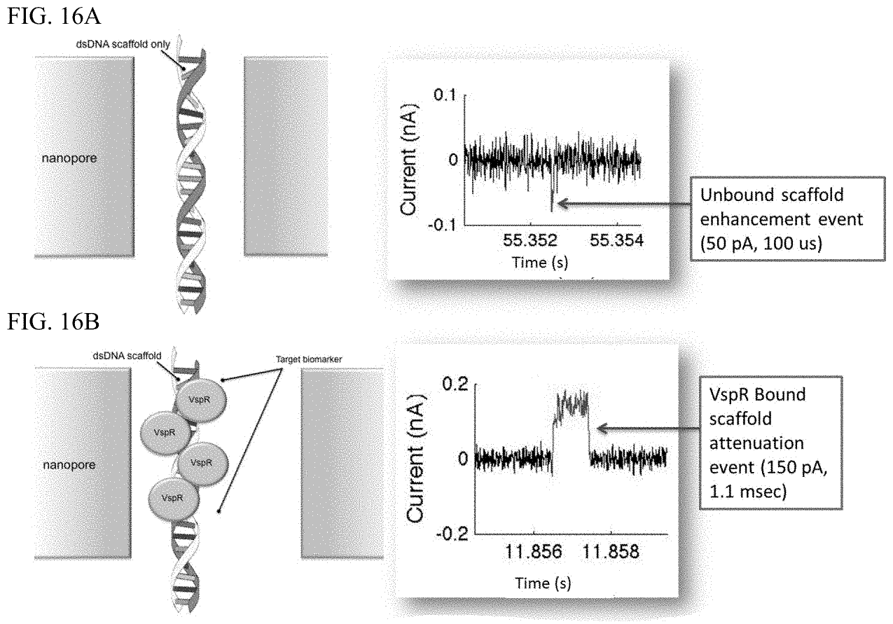

The VspR protein is a 90 kDa protein from V. cholerae that binds directly to dsDNA with high micromolar affinity in a sequence specific manner (see reference: Yildiz, Fitnat H., Nadia A. Dolganov, and Gary K. Schoolnik. “VpsR, a Member of the Response Regulators of the Two-Component Regulatory Systems, Is Required for Expression of Biosynthesis Genes and EPSETr-Associated Phenotypes in Vibrio cholerae O1 El Tor.” Journal of bacteriology 183, no. 5 (2001): 1716-1726). In this example of target sequence detection using nanopore technology, VspR acts as the probe molecule with a site-specific DNA binding domain. In this experiment, we showed detection of the VspR on the DNA scaffold as a model of using a protein to detect a specific sequence in DNA is present. The DNA scaffold contained 10 VspR specific binding sites (FIG. 15). To preserve affinity of VspR for dsDNA binding, we used 0.1 M KCl, a salt concentration in which VspR-bound scaffold translocation through the nanopore enhances current flow through the nanopore (FIG. 16). We provided a solution containing VspR protein at a concentration of 18 nM in the recording buffer, and 180 nM during labeling (binding step). This results in 18× excess of VspR protein to binding sites on DNA. The experiment was run at pH 8.0 (pI of VspR protein is 5.8). Taking Kd and DNA concentration into account, only 0.1-1% of DNA should be fully occupied by VspR, with a larger percentage partially occupied, and some unknown remaining percentage of DNA entirely unbound. There is also free VspR protein in solution during the nanopore experiment.

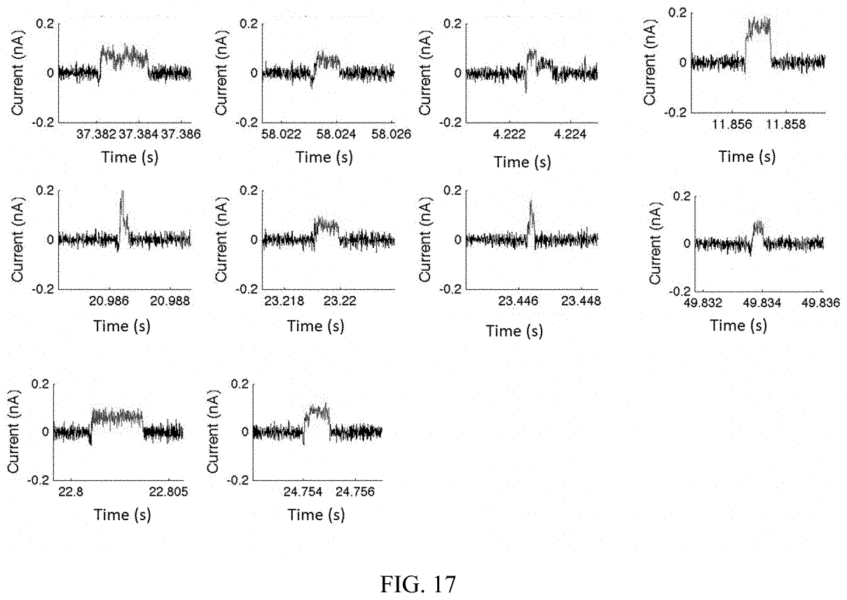

Two representative events are shown in FIG. 16A and FIG. 16B. In the experiments with VspR, VspR concentration was 18 nM (1.6 mg/L), 10 nM binding sites. The scaffold concentration was 1 nM resulting in capture every 6.6 seconds. The pore size is 15 nm in diameter and length. The voltage is −100 mV, and note that negative voltages create negative currents, so upward shifts correspond to attenuation events, as shown for the VspR-bound DNA event (FIG. 16B), whereas downward shifts create positive shifts as shown for the unbound DNA scaffold event (FIG. 16A). This is consistent with the idealized signal patterns and conditions in FIG. 2, with the DNA event (FIG. 16A) having faster duration and the opposite polarity compared to the fusion molecule-bound DNA event (FIG. 16B). Thus, the key observation from this figure is that VspR-bound events have the opposite signal polarity compared to unbound DNA events and therefore easily detectable indication that the specific DNA sequence is present. FIG. 17 shows ten more representative current attenuation events consistent with the VspR-bound scaffold passing through the pore. There were 90 such events over 10 minutes of recording, corresponding to 1 VspR-bound event every 6.6 seconds. Events were attenuations of 50 to 150 pA in amplitude and 0.2 to 2 milliseconds in duration. As stated, downward events correspond to current enhancement events and upward events correspond to current attenuation events in FIGS. 16-17, and this shift direction is preserved even though the baseline is zeroed for display purposes.

### Example 4—Sequence Specific Probe Synthesis and Target Sequence Binding

In this example, we show the generation of a PNA probe for binding to a target sequence of interest, with features added to the PNA probe to allow for increased sensitivity of detection in a nanopore.

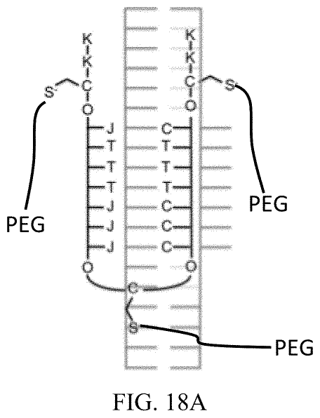

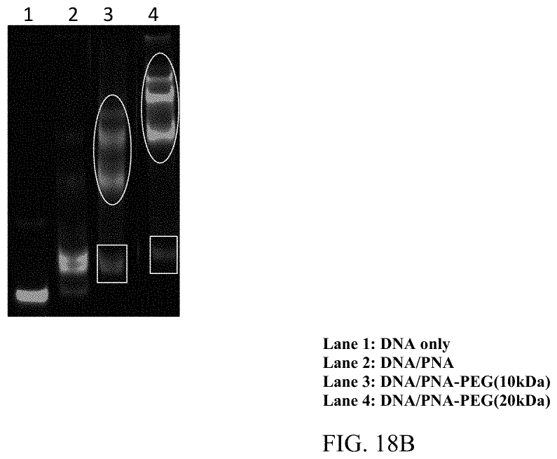

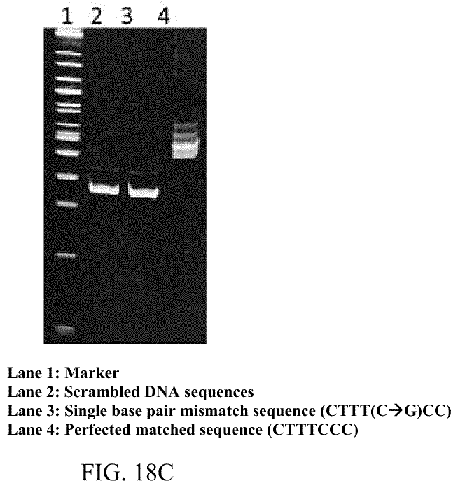

We generated a bisPNA probe containing 3 cysteine residues. The bisPNA probe comprises a sequence of PNA capable of binding to its a DNA sequence comprising a target sequence of CTTTCCC at the location of this target sequence on a target DNA molecule. The bisPNA probe was also labeled with maleimido-PEG-Me at 3 cysteine residues on the bisPNA probe to enhance detection of the probe attached to a target DNA molecule in a nanopore. The PNA-PEG probe was generated by incubating a 100 fold excess of linker (Methyl-PEG (10 kDa)-Maleimide) with bisPNA (Lys-Lys-Cys-PEG3-JTTTJJJ-PEG-Cys-PEG-CCCTTTC-PEG-Cys-Lys-Lys) under reducing conditions. The maleimide portion of the linker reacts with the free thiols in the PNA at pH 7.4, thus creating the PEGylated-PNA. The addition of lysines increases the reagent affinity for its specific cognate DNA sequence thereby allowing it to remain bound under high salt conditions (1 M LiCl). The resulting PNA-PEG probe bound to its target sequence on a dsDNA molecule is shown in FIG. 18A.

To confirm the binding of the DNA-PEG probe to its target sequence on a DNA molecule, we incubated different versions of the PEG probe with DNA and performed a gel shift assay using the resulting solution. For this assay, we ran 4 samples, as shown in FIG. 18B. Lane 1 is DNA only, lane 2 is DNA+PNA, lane 3 is DNA+PNA-PEG (10 kDa), and lane 4 is DNA+PNA-PEG (20 kDa). The upward shift in lanes 2-4 is consistent with the bisPNA species being bound to DNA. The circled species are DNA/PNA-PEG, the boxed species are DNA/PNA in lanes 3 and 4 present as residual PNA (sans PEG) in the labeling experiment. The results of the gel shift assay show complex formation of a DNA containing the target sequence and the PNA probe with a cognate DNA sequence complementary to the target sequence regardless of the attachment of a PEG to the PNA. Thus, we here show successful complex formation of a sequence-specific probe capable of being detected in a nanopore.

We then performed an assay to show specificity of the PNA probe for its target DNA sequence. Here, we incubated a PNA probe (without PEG), with a sample comprising DNA without the target sequence (lane 2), DNA with the target sequence comprising a single base mismatch with the PNA probe (lane 3), and DNA with the complete target sequence (lane 4), and analyzed each sample using a gel shift assay, the results of which are shown in FIG. 18C. Lane 1 is a DNA marker. As shown by our results, DNA with an exact target sequence match (lane 4) binds the PNA, while DNA with the target sequence comprising a single base mismatch sequence (lane 3), and DNA without the target sequence (random sequence in place of the target sequence) (lane 2) show no PNA binding. Therefore, the gel shift assay shows that PNA specifically binds to DNA comprising its target sequence, without binding to DNA having even a single mismatch in the target sequence.

### Example 5—Target Sequence Detection in a Nanopore Using a Modified Sequence-Specific Probe

In this example, we show detection of a DNA molecule comprising the target sequence bound to our PEG modified sequence-specific PNA probe.

Here we provided three different PNA probes to have different bulkiness based on PEG attachment and PEG length. Three types of probes were used: 1) PNA with no PEG, 2) PNA bound to a 5 kDa PEG, and 3) PNA bound to a 10 kDa PEG. Each probe was mixed with DNA comprising the target sequence and run in the nanopore to observe detection of the DNA bound to the PNA probe. The concentration of each complex in the sample was 2 nM in 1M LiCl buffer. The sample was run in the nanopore device under an applied voltage of 100 mV. The results are shown in FIGS. 19A-19E.

Representative individual events observed are shown for DNA bound to each type of probe in FIG. 19A. The event signature from a DNA/bisPNA event is shown on the left. The event signature from a DNA/bisPNA-PEG complex with up to 3 PEGs bound to each PNA, and PEG sized 5 kDa is shown in the middle. The event signature from a DNA/bisPNA-PEG complex with up to 3 PEGs bound to each PNA, and PEG sized 10 kDa is shown on the right. The event signature for each was measured by current blockade through the nanopore during translocation of the identified complex. In FIG. 19A, molecule depictions show linear PEG and DNA sized to scale for visual comparison. As the probe size (bulk) is increased, the event signature changes.

We analyzed the population of events and generated a scatter plot of mean conductance shift (dG) vs. duration for all events in each data set. We generated a scatter plot of event mean conductance (mean current shift divided by voltage) versus event duration (width) from our experiment as shown in FIG. 19B. The plot shows that DNA/PNA, DNA/PNA-PEG (5 kDa), and DNA/PNA-PEG (10 kDa) give overlapping populations that are distinct based on their event duration and mean conductance. We generated a histogram to show the observed difference in mean conductance shift for each event (dG) between the different complexes (FIG. 19C). We also generated a histogram to show the observed difference in event duration between the different complexes (FIG. 19D).

### Example 6—Detection of a Mutated Cftr Gene Target Sequence in a Nanopore to Detect Human Cystic Fibrosis

We have shown the specificity of binding of our modified PNA probe to a target sequence and the ability to detect the target sequence using the probe in a nanopore device. Here, we look to the use of the modified PNA probe in a nanopore device to detect a disease causing mutation in a sample from a patient, specifically cystic fibrosis.

We generated (according to the methods described in Example 4) a modified PNA probe (PNA-PEG probe) which comprises a PNA molecule that binds specifically to a target DNA sequence comprising a cftr gene with a mutation therein (ΔF508) which causes cystic fibrosis. The PEG bound to the PNA probe was 5 kDa. DNA containing a Cystic Fibrosis disease mutation was incubated with a PEGylated PNA specific for the mutation. The samples were then placed in a nanopore device having a 26 nm pore and translocation events through the nanopore were recorded and analyzed.

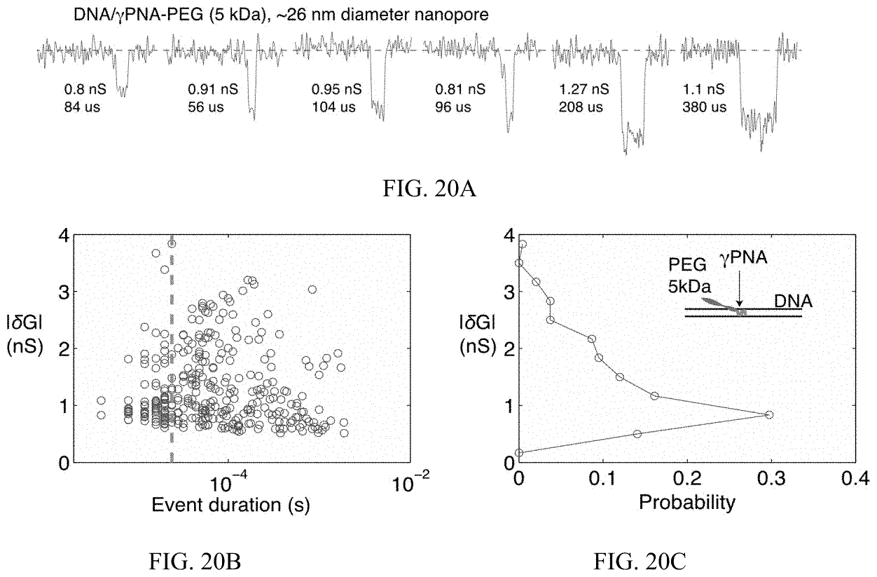

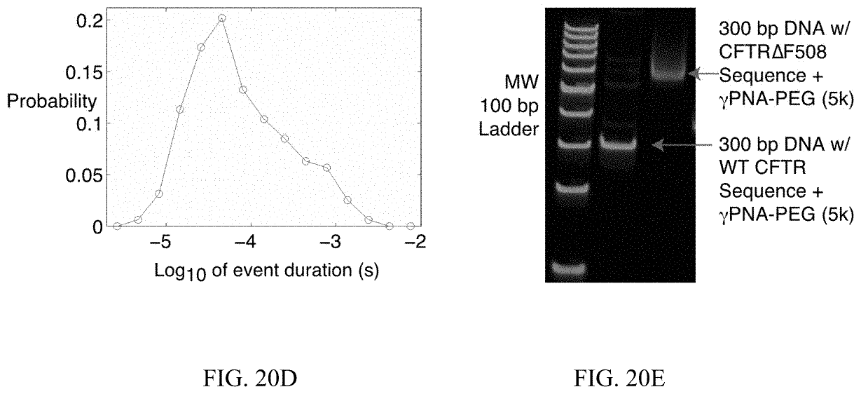

Translocation event signatures correlated with the translocation of a PNA-PEG probe bound to a DNA molecule were observed in the sample with DNA containing the cystic fibrosis causing mutation (ΔF508). Representative event signatures are shown in FIG. 20A. Experiments using sample with DNA only or DNA/PNA only (i.e., no PEG-PNA) gave no definitive translocation events above background, showing the ability of the pore to accurately identify PNA-PEG probe bound to DNA, and the enhancement of detection provided by the modified probes provided herein. For the set of recorded events from a sample with the target mutated gene and the PNA-PEG probe, the events were characterized by mean conductance shift and duration and analyzed. FIG. 20B shows the mean conductance shift v. duration plot for each recorded event. FIG. 20C and FIG. 20D show corresponding histograms to characterize the events detected by mean conductance shift and duration of each event respectively. The analyzed data matched the expected data for a DNA/PNA-PEG (5 kDa) complex translocation through the nanopore, indicating successful binding and identification of the cftr mutation target sequence in the nanopore device.

We also ran a gel shift assay on samples comprising our PNA-PEG (5 kDa) probe specific for the ΔF508 cftr gene mutation with a sample comprising 300 bp DNA with the wild-type cftr sequence (lane 2) and with a sample comprising 300 bp DNA with the ΔF508 cftr gene mutation (lane 3) (FIG. 20E). This data shows that our PNA-PEG probe binds specifically to only the ΔF508 target sequence, but does not bind to the wild-type sequence. Therefore, we have successfully detected DNA comprising the single base cftr gene mutation (ΔF508), and have here demonstrated the use of our system to detect specific sequences of a polynucleic acid in a sample, including for diagnostic or treatment indications in a human patient.

### Example 7—Infectious Bacteria Detection with the PNA-PEG Probe in a Nanopore

In this example, we look at the use of our modified probes to detect the presence of bacterial DNA in a sample using a nanopore device.

We synthesized a probe with a PNA molecule capable of specifically binding to Staphylococcus mitis (S. mitis) bacterial DNA. The bisPNA contains a sequence complementary to a sequence that is specific for the S. mitis bacteria species.

In this assay, the PNA probe is bound to 10 kDa PEG to allow for detection in a nanopore when bound to the bacterial DNA. We mixed the PNA probe with the bacterial DNA and performed a gel shift assay on the sample to observe binding. FIG. 21A shows the results of the gel shift assay, with lane 1 comprising bacterial DNA without the PNA probe, and lane 2 comprising bacterial DNA with the PNA probe. Our observed results show that our PNA/PEG (10 kDa) probe bound to the S. mitis bacterial DNA.

We next prepared two samples for detection in a nanopore. The first sample included bacterial DNA with PEG-modified PNA probes (DNA/bisPNA-PEG). The second sample included bacterial DNA alone. We ran these samples through a nanopore device in two consecutive experiments, and analyzed the resulting events. FIG. 21B shows a scatter plot of mean conductance shift (dG) on the vertical axis vs. duration on the horizontal axis for all recorded events in the two consecutive experiments. Events characterized by from tagged sample 1 (squares) and untagged sample 2 (circles) are shown.

The tagged molecules are consistently above a background threshold (dashed line), while untagged molecules are below the line and consistent with a background population. The population of molecules from a variety of background experiments (DNA/PNA without PEG, filtered serum, etc.) are used to establish the threshold (line) for flagging tagged events. Background events are not shown here. For accurate detection of bacterial DNA in a sample, the DNA must be tagged using a highly site-specific probe.

Our results show that the PNA/PEG bound population of S. mitis bacterial DNA is discernable from background events while DNA only and DNA/PNA only are not. Thus, the our modified PNA-PEG sequence specific probe allows confident detection of the presence or absence of S. mitis DNA in a sample.

### Other Embodiments

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A method of detecting a polynucleotide comprising a target sequence in a sample, the method comprising:
a) contacting said sample with a probe that specifically binds to said polynucleotide comprising said target sequence under conditions that promote binding of said probe to said target sequence to form a polynucleotide-probe complex;
b) loading said sample into a first chamber of a nanopore device, wherein said nanopore device comprises at least one nanopore and at least said first chamber and a second chamber, wherein said first and second chamber are in electrical and fluidic communication through said at least one nanopore, and wherein the nanopore device further comprises an independently-controlled voltage across each of said at least one nanopores and a sensor associated with each of said at least one nanopores, wherein said sensor is configured to identify objects passing through the at least one nanopore, and wherein said polynucleotide-probe complex translocating through said at least one nanopore provides a detectable signal associated with said polynucleotide-probe complex; and
c) determining the presence or absence of said polynucleotide-probe complex in said sample by observing said detectable signal, thereby detecting said polynucleotide comprising said target sequence.

2. The method of claim 1, wherein said polynucleotide is DNA or RNA.

3. The method of claim 1, wherein said detectable signal is an electrical signal.

4. The method of claim 1, wherein said detectable signal is an optical signal.

5. The method of claim 1, wherein said probe comprises a molecule selected from the group consisting of: a protein, a peptide, a nucleic acid, a TALEN, a CRISPR, a peptide nucleic acid, or a chemical compound.

6. The method of claim 1, wherein said probe comprises a molecule selected from the group consisting of: a deoxyribonucleic acid (DNA), a ribonucleic acid (RNA), a peptide nucleic acid (PNA), a DNA/RNA hybrid, polypeptide, or any chemically derived polymer.

7. The method of claim 1, wherein said probe comprises a PNA molecule bound to a secondary molecule configured to facilitate detection of the probe bound to said polynucleotide during translocation through said at least one nanopore.

8. The method of claim 7, wherein said secondary molecule is a PEG.

9. The method of claim 8, wherein said PEG has a molecular weight of at least 1 kDa, 2 kDa, 3 kDa, 4 kDa, 5 kDa, 6 kDa, 7 kDa, 8 kDa, 9 kDa, or 10 kDa.

10. The method of claim 1, further comprising applying a condition to said sample suspected to alter the binding interaction between the probe and the target sequence.

11. The method of claim 10, wherein the condition is selected from the group consisting of: removing the probe from the sample, adding an agent that competes with the probe for binding to the target sequence, and changing an initial pH, salt, or temperature condition.

12. The method of claim 1, wherein said polynucleotide comprises a chemical modification configured to modify binding of the polynucleotide to the probe.

13. The method of claim 12, wherein the chemical modification is selected from the group consisting of biotinylation, acetylation, methylation, summolation, glycosylation, phosphorylation and oxidation.

14. The method claim 1, wherein said probe comprises a chemical modification coupled to the probe through a cleavable bond.

15. The method of claim 1, wherein the probe interacts with the target sequence of the polynucleotide via a covalent bond, a hydrogen bond, an ionic bond, a metallic bond, van der Waals force, hydrophobic interaction, or planar stacking interactions.

16. The method of claim 1, further comprising contacting the sample with one or more detectable labels capable of binding to the probe or to the polynucleotide-probe complex.

17. The method of claim 1, wherein the polynucleotide comprises at least two target sequences.

18. The method of claim 1, wherein the nanopore is about 1 nm to about 100 nm in diameter, 1 nm to about 100 nm in length, and wherein each of the chambers comprises an electrode.

19. The method of claim 1, wherein said nanopore device comprises at least two nanopores configured to control the movement of said polynucleotide in both nanopores simultaneously.

20. The method of claim 1, further comprising reversing said independently-controlled voltage after initial detection of the polynucleotide-probe complex by said detectable signal, so that the movement of said polynucleotide through the nanopore is reversed after the probe-bound portion passes through the nanopore, thereby identifying again the presence or absence of a polynucleotide-probe complex.

21. The method of claim 1, wherein said nanopore device comprises two nanopores, and wherein said polynucleotide is simultaneously located within both of said two nanopores.

22. The method of claim 21, further comprising adjusting the magnitude and or the direction of the voltage in each of said two nanopores so that an opposing force is generated by the nanopores to control the rate of translocation of the polynucleotide through the nanopores.

23. A method of detecting a polynucleotide or a polynucleotide sequence in a sample, comprising:
a) contacting said sample with a first probe and a second probe, wherein said first probe specifically binds to a first target sequence of said polynucleotide under conditions that promote binding of said first probe to said first target sequence, wherein said second probe specifically binds to a second target sequence of said polynucleotide under conditions that promote binding of said second probe to said second target sequence;
b) contacting said sample with a third molecule is configured to bind to said first and second probe simultaneously when said first and second probe are within a sufficient proximity to each other under conditions that promote binding of said third molecule to said first probe and said second probe, thereby forming a fusion complex comprising said polynucleotide, said first probe, said second probe, and said third molecule;
c) loading said sample into a first chamber of a nanopore device, wherein said nanopore device comprises at least one nanopore and at least said first chamber and a second chamber, wherein said first and second chamber are in electrical and fluidic communication through said at least one nanopore, and wherein the nanopore device further comprises a controlled voltage potential across each of said at least one nanopores and a sensor associated with each of said at least one nanopores, wherein said sensor is configured to identify objects passing through the at least one nanopore, and wherein said fusion complex translocating through said at least one nanopore provides a detectable signal associated with said fusion complex; and
d) determining the presence or absence of said fusion complex in said sample by observing said detectable signal.

24. The method of claim 23, wherein said polynucleotide is DNA or RNA.

25. The method of claim 23, wherein said detectable signal is an electric signal.

26. The method of claim 23, wherein said detectable signal is an optical signal.

27. The method of claim 23, wherein said sufficient proximity is less than 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 300, 400, or 500 nucleotides.

28. The method of claim 23, wherein said third molecule comprises a PEG or an antibody.

29. The method of claim 23, wherein said third molecule and said first and second probes are bound to ssDNA, and wherein said ssDNA linked to said third molecule comprises a region complementary to a region of ssDNA linked to said first probe and is complementary to a region of ssDNA linked to said second probe.

30. The method of claim 23, further comprising contacting the sample with one or more detectable labels capable of binding to the third molecule or to the fusion complex.

31. A kit comprising a first probe, a second probe, and a third molecule, wherein the first probe is configured to bind to a first target sequence on a target polynucleotide, wherein the second probe is configured to bind to a second target sequence on said target polynucleotide, and wherein said third molecule is configured to bind to the first probe and the second probe when said first and second probes are bound to said polynucleotide at said first and second target sequences, thereby locating the first and second probe in sufficient proximity to allow binding of said third molecule to said first and second probes simultaneously.

32. The kit of claim 31, wherein said first probe and said second probe are selected from the group consisting of: a protein, a peptide, a nucleic acid, a TALEN, a CRISPR, a peptide nucleic acid, or a chemical compound.

33. The kit of claim 31, wherein said third molecule comprises a PEG or an antibody.

34. The kit of claim 31, wherein said third molecule comprises a modification to modify binding affinity to said probes.

35. A nanopore device comprising at least two chambers and a nanopore, wherein said device comprises a modified PNA probe bound to a polynucleotide within said nanopore.

36. A dual-pore, dual-amplifier device for detecting a charged polymer through two pores, the device comprising an upper chamber, a middle chamber and a lower chamber, a first pore connecting the upper chamber and the middle chamber, and a second pore connecting the middle chamber and the lower chamber, wherein said device comprises a modified PNA probe bound to a polynucleotide within said first or second pore.

37. The device of claim 36, wherein said device is configured to control the movement of said charged polymer through both said first pore and said second pore simultaneously.

38. The device of claim 36, wherein said modified PNA probe is bound to at least one PEG molecule.

39. The device of claim 36, wherein the device further comprises a power supply configured to provide a first voltage between the upper chamber and the middle chamber, and provide a second voltage between the middle chamber and the lower chamber, each voltage being independently adjustable, wherein the middle chamber is connected to a common ground relative to the two voltages, wherein the device provides dual-amplifier electronics configured for independent voltage control and current measurement at each pore, wherein the two voltages may be different in magnitude, wherein the first and second pores are configured so that the charged polymer is capable of simultaneously moving across both pores in either direction and in a controlled manner.
